(19) United States
(12) Patent Application Publication
(10) Pub. No.: US 2023/0081679 A1
(43) Pub. Date: Mar. 16, 2023

(54) SURFACTANT VESICLES FOR VACCINE FORMULATION, TARGETED DRUG DELIVERY, AND TRANSFECTION

(71) Applicant: University of Maryland, College Park, College Park, MD (US)

(72) Inventors: Philip R. DESHONG, Silver Spring, MD (US); David WATTS, San Mateo, CA (US); Daniel C. STEIN, Silver Spring, MD (US); Paul PAUKSTELIS, Silver Spring, MD (US); Emily LUTERAN, Adelphi, MD (US)

(21) Appl. No.: 17/929,630

(22) Filed: Sep. 2, 2022

Related U.S. Application Data
(60) Provisional application No. 63/240,289, filed on Sep. 2, 2021.

(51) Int. Cl. A61K 9/127; C07K 16/30; C07K 16/28; A61K 39/39; C12N 15/113

(52) U.S. Cl. CPC A61K 9/1272; A61K 9/1277; C07K 16/3015; C07K 16/2887; C07K 16/2818; A61K 39/39; C12N 15/1135; C12N 15/1138; A61K 2039/55555

(57) ABSTRACT

The present disclosure provides catanionic surfactant vesicles (SVs). The vesicles may be functionalized on their outer leaflet such that they may be biologically active. The vesicles may encapsulate (at least partially in the lumen and/or at least partially in the leaflet) one or more small molecules, one or more RNAs, one or more DNAs, and/or one or more proteins/peptides. Also provided are compositions comprising the vesicles (e.g., vaccine compositions comprising the vesicles) and methods of making and using the same.

### CROSS REFERENCE TO RELATED APPLICATIONS

This application claims priority to U.S. Provisional Application No. 63/240,289, filed on Sep. 2, 2021, the disclosure of which hereby is incorporated herein by reference in its entirety.

### STATEMENT REGARDING FEDERALLY SPONSORED RESEARCH

This invention was made with government support under R01AI123340A awarded by the National Institutes of Health (NIH). The government has certain rights in the invention.

### SEQUENCE LISTING

The instant application contains a Sequence Listing which has been submitted in .xml format and is hereby incorporated by reference in its entirety. Said .xml copy was created on Aug. 31, 2022, is named “070919_00114_ST26.xml”, and is 5,794 bytes in size.

### BACKGROUND OF THE DISCLOSURE

Nanoparticle based drug delivery systems have proven to be an important advance in the drug delivery field. Doxil, a liposomal formulation of doxorubicin, was the first liposome-drug approved for clinical use because of its ability to decrease the cardiotoxicity associated with doxorubicin administration. Doxil is the most widely used oncology chemotherapeutic in the world. Subsequently, a variety of nanoparticle formulations employing liposomes and niosomes have been approved for clinical use. However, liposome-based formulations as a generalized drug delivery vehicle face a series of issues: (1) control of the size distribution of the nanoparticle, (2) stability of the nanoparticles in buffers and biological media, (3) stability of the formulations for long term storage, and (4) inability to incorporate effective targeting strategies.

Catanionic surfactant vesicles (SVs) have emerged as an attractive alternative to liposomes due to their robust chemophysical properties. SVs are formed from mixtures of two surfactants with single alkyl tails and oppositely charged head groups. Catanionic SVs offer several advantages over conventional liposomes which are made from double-tailed zwitterionic phospholipids: (1) inexpensive starting materials, (2) spontaneous formation of vesicle structures with a diameter of 160±30 nm, (3) increased stability in buffer and complex aqueous media, (4) ability to control zeta potential (surface charge) based on the ratio of surfactants employed, and (5) ability to decorate the outer surface of the leaflet with a wide variety of targeting moieties.

### SUMMARY OF THE DISCLOSURE

The present disclosure generally relates to the production of highly functionalized surfactant vesicles including a wide variety of molecules with relevance to vaccine formulation, targeted drug delivery, and transfection. Examples of the present disclosure demonstrate that surfactant vesicles can be utilized to encapsulate a variety of carbohydrates, nucleic acid derivatives (e.g., RNA and DNA), and chemotherapeutic agents. Examples of the present disclosure also show that resulting vesicle formulations with the encapsulated components are robust and can remain stable at room temperature for extended periods under a wide variety of conditions. Further, examples of the present disclosure show that the therapeutic agents encapsulated in such vesicles can be delivered to cells whereby the vesicles release their nucleic acid contents into the cells.

### BRIEF DESCRIPTION OF THE FIGURES

For a fuller understanding of the nature and objects of the disclosure, reference should be made to the following detailed description taken in conjunction with the accompanying figures.

FIG. 1. Schematic representation of an anion rich catanionic SV. Dark gray circles are cationic surfactant heads, light gray circles are anionic surfactant heads, triangles represent various targeting moieties tethered to the bilayer by hydrophobic tails, ellipses are hydrophobic drugs embedded in the bilayer, and hexagons are hydrophilic drugs trapped within the lumen.

FIG. 2. Mean radius, from DLS, of catanionic SVs with 60:40 w/w surfactant ratio prepared in 0.5 M glucose solution after gel filtration to remove excess unencapsulated sugar. Empty circles—vesicles prepared in PBS 1×. Filled squares—vesicles prepared in deionized water.

FIG. 3. Vesicles prepared in 0.1, 0.2, and 0.5 M glucose solutions. Glucose concentration in purified vesicle solutions (squares) and EE (circles) is shown. Error bars represent the standard deviation of three separate batches of vesicles. Glucose concentrations after purification were analyzed by ¹H-NMR.

FIG. 4. Vesicle size as measured by DLS over time when stored in dialysis tubing (1 kD MWCO, regenerated cellulose) at room temperature. Each data point is an average of three measurements while error bars represent the size distribution as approximated from the average of the PDI's for each measurement.

FIG. 5. Plot of the natural log of starting glucose concentration vs natural log of the initial rate of glucose release from catanioinc SVs yields a linear fit with slope of 1.1±0.2, suggesting diffusion is first order in glucose concentration. Error bars represent the standard deviation derived from triplicate analysis of glucose-SVs.

FIG. 6. Release rate profiles of three different batches of vesicle encapsulated glucose along with the predicted [Gluc]/[Gluc]ₜ₋₀ from pseudo-first order kinetics (dotted line). Error bars are derived from three separate batches of vesicle encapsulated glucose preparations.

FIG. 7. Release profiles for the disaccharides ((A) D-maltose, (B) D-cellobiose, (C) D-sucrose) and (D) maltotriose. First order fits to the first part of each experiment (t=0-100 h) showed poor correlation to the remainder (t=100-500 h) of each experiment. All release experiments were carried out at 25° C. in PBS 1× using 60:40 w/w SDBS:CTAT surfactant ratios and 1% w/w total surfactant in solution.

FIG. 8. The conversion of carboplatin to cis-[Pt(NH₃)₂(CBDCA-O)Cl]Na (CBDCA=cyclobutene-1,1-dicarboxylate), free CBDCA, and cisplatin in PBS 1× at 25° C.

FIG. 9. 20% denaturing PAGE gels showing (A)—Encapsulation and protection of ssDNA (13 nt) by catanionic vesicles. Vesicles and free ssDNA were challenged with nuclease (51 Nuclease, 0.5 U, rt, 0.5 h). Vesicles were disrupted by three freeze/thaw cycles either with or without nuclease present and with or without centrifugation at 10,000 rpm for 5 min to remove excess surfactant material after disruption. (B)—Encapsulation and protection of siRNA (21 bp) by catanionic vesicles. Vesicles and free siRNA (133 μg/mL) were challenged with RNase (Type X11-A, 10 U, rt, 0.5 h). Vesicles were disrupted by three freeze/thaw cycles.

FIG. 10. Vesicle-incorporated DNA duplexes are protected from nuclease digestion. Vesicle-incorporated fluorescently-quenched 20 bp DNA duplexes were subjected to DNase I cleavage at 25° C. (A) or 37° C. (B) before (intact) and after (broken) freeze/thaw cycles to promote vesicle rupturing. Results show three independent samples for the ruptured vesicles and the average and standard deviation for three intact vesicle samples. Variation in overall fluorescent intensity in the ruptured samples reflect the variability in vesicle rupture and reformation during the freeze/thaw process.

FIG. 11. Agarose gel showing A: Protection of every band in a 10 kbp DNA ladder by catanionic vesicles. B: Increased vesicle encapsulation of DNA pre-treated with spermine. Conditions for DNase treatment are 10 U/mL TURBO DNase, 0.5 mM CaCl₂), 2.5 mM MgCl₂, in PBS 1× at 37° C. for 1 h. Conditions for vesicle disruption: 50 μL of vesicle solution is mixed with 20 μL 0.5 g/mL HPβCD (2-hydroxypropyl-β-cyclodextrin) in water and vortexed.

FIG. 12. Agarose gel showing protection of pUC18 and GFP-Plasmid by SVs. pUC18-SV and GFP-SV represent vesicle encapsulated plasmid formulations while pUC18 and GFP-SV represent free plasmid solutions. Conditions for DNase treatment are 10 U/mL TURBO DNase, 0.5 mM CaCl₂), 2.5 mM MgCl₂, in PBS 1× at 37° C. (a, b) Vesicle encapsulated pUC18 plasmid, incubated with DNase for 4 and 18 h, respectively. (c) pUC18 mixed with bare vesicles. (d) pUC18. (e) Linearized pUC18, prepared by treatment with EcoRI, showing that the larger mw bands in the other lanes do not correspond to linearized pUC18. (f) GFP-Plasmid. (g) vesicle encapsulated GFP-plasmid. (h) GFP-plasmid incubated with TURBO DNase for 1h (i, j, k) GFP-plasmid incubated with TURBO DNase for 1, 4 and 22 h respectively. (g) Vesicle encapsulated GFP plasmid broken up with HPβCD in the presence of TURBO DNase, incubated for 1 h.

FIG. 13. Agarose gel showing some protection of linearized 9230 bp plasmid DNA. Conditions for DNase treatment are 10 U/mL TURBO DNase, 0.5 mM CaCl₂), 2.5 mM MgCl₂, in PBS 1× at 37° C. for 1 h. Conditions for vesicle disruption: 50 μL of vesicle solution is mixed with 20 μL 0.5 g/mL HPβCD (2-hydroxypropyl-β-cyclodextrin) in water and vortexed.

FIG. 14. Schematic representation highlighting the various modes of payload delivery. Left—intact catanionic vesicles may be functionalized on the surface with biological molecules that feature a hydrophobic moiety. Right—vesicles may trap hydrophilic payloads in the lumen or hydrophobic payloads in the leaflet. Circles are cationic surfactant heads and anionic surfactant heads.

FIG. 15. Comparison of phospholipid liposomes to catanionic vesicles, highlighting beneficial catanionic vesicle properties. Specifically, spontaneous vesicle formation and thermostability is highlighted. Cryo-EM shows catanionic vesicles are unilamellar and DLS depicts a tight vesicle size distribution with a PDI of 0.164.

FIG. 16. Comparison of catanionic vesicle chemistry to the primary competing technology, antibody-drug conjugation. Superior catanionic vesicle characteristics include ease of preparation, diversity of targeting agents, and greater drug loading capacity.

FIG. 17. Schematic showing the general synthetic scheme to produce catanionic vesicle encapsulated drug formulations (left). Kinetic data (right) showing hydrophilic drugs (pemetrexed, carboplatin, cisplatin) are released with half-lives ranging from 1-50 h while the hydrophobic drug doxorubicin retained within the vesicle for a much longer time.

FIG. 18. Fluorescence microscopy showing the targeted delivery of doxorubicin loaded catanionic vesicles decorated with a folate-lipid conjugate to A549 human cancer cells. Left—overlay bright field and fluorescence (excitation 480, emission 590) images of A549 cells prior to inoculating with vesicles. Right—overlay of bright field and fluorescence (excitation 480, emission 590) images of A549 cells 1 hour post inoculation with vesicles, showing accumulation of doxorubicin within the cell nuclei.

FIG. 19. Agarose gel showing protection of GFP-encoding plasmid against DNase by SVs. Conditions for DNase treatment are 10 U/mL TURBO DNase, 0.5 mM CaCl₂), 2.5 mM MgCl₂, in PBS 1× at 37° C. (a) GFP plasmid. (b) Vesicle encapsulated GFP plasmid. (c) GFP plasmid incubated with DNase for 1 h. (d, e, f) Vesicle encapsulated GFP plasmid incubated with DNase for 1, 4, and 22 h. (g) Vesicle encapsulated GFP plasmid broken up with HPβCD in the presence of DNase, incubated for 1 h.

FIG. 20. Schematic for incorporation of the DNA-lipid conjugate and fluorescent data of SVs with fluorescently tagged DNA.

FIG. 21. Schematic for encapsulation of a bioactive enzyme (horseradish peroxidase (HRP)). Vials (top row) show HRP catalyzed oxidation of phenol red over time. Vials (bottom row) show the same vesicles without HRP encapsulated. A decrease in absorbance of phenol red is shown and indicates its oxidation.

FIG. 22. General synthetic scheme for producing folate targeted doxorubicin loaded catanionic vesicles. SDBS and CTAT are combined in a solution containing the depicted folate conjugate as well as doxorubicin, both of which spontaneously imbed in the leaflet with folate exposed to bulk solution. Dark gray spheres are both cationic and anionic surfactant heads, light gray spheres are doxorubicin, and squares are folate moieties.

FIG. 23. General synthetic scheme for producing antibody targeted catanionic vesicles. Vesicles which are surface decorated with a cyclooctyne moiety are mixed with a solution containing an antibody-azide conjugate, which spontaneously react to form a triazole linkage, tethering the antibody to the vesicle surface.

FIG. 24. Protein gel demonstrating successful conjugation of azido-modified antibodies (Rituximab-N₃ and Herceptin-N₃) with cyclooctyne decorated vesicles.

FIG. 25. Comparison of carboxyfluorescein release as a function of time, R(t), between cation rich catanionic vesicles (solid line) and phospholipid vesicles (dotted line). Release of carboxyfluorescein has an extrapolated half-life of 84 days vs 2 days in phospholipid vesicles.

FIG. 26. Fluorescence microscopy images highlighting selectivity of targeted catanionic vesicles. Top image—A549 cells. Middle image—A549 cells that have been inoculated with doxorubicin loaded vesicles that do not feature any cell targeting functionality. Bottom image—A549 cells that have been inoculated with doxorubicin loaded vesicles decorated with a folate-lipid conjugate, showing much higher payload delivery.

FIG. 27. IC₅₀ values for surfactant vesicles and surfactant vesicles containing doxorubicin in A549 and Igrov 1 cells.

FIG. 28. Weight variation of mice given bare vesicles and doxorubicin vesicles over a period of 24 days.

FIG. 29. Treatment of vesicles containing lipid oligosaccharide (LOS) from Neisseria gonorrhoeae with beta-galactosidase results in truncation of the LOS. The LOS can be reconstituted by treatment of the vesicle with UDP-galactose and galactosyltransferase.

FIG. 30. ¹H-NMR spectrum (DMSO-d₆, 600 MHz) of anion rich catanionic vesicles that have been purified by gel filtration. Of note is the lack of tosylate, demonstrating that gel filtration is an adequate method of purification.

FIG. 31. Schematic representing spontaneous vesicle functionalization. When intact vesicles are incubated with any lipid conjugate, the hydrophobic portion embeds in the lipid bilayer, providing an easy and simple surface modification technique.

FIG. 32. Schematic highlighting the modularity of catanionic vesicle surface functionalization. The surface ligand number density, mode of presentation, and number of different epitopes can be adjusted by changing the anion/cation ratio, ligand concentration, and number of different functionalizing molecules in solution.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

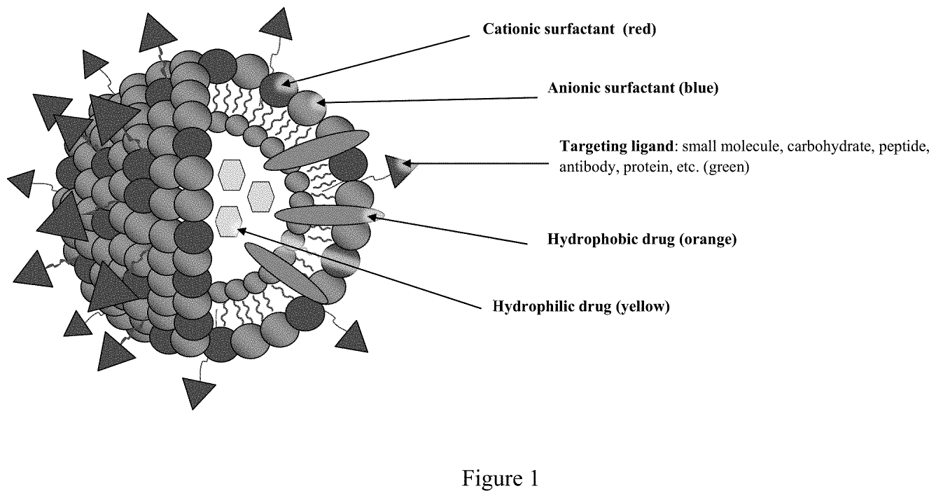

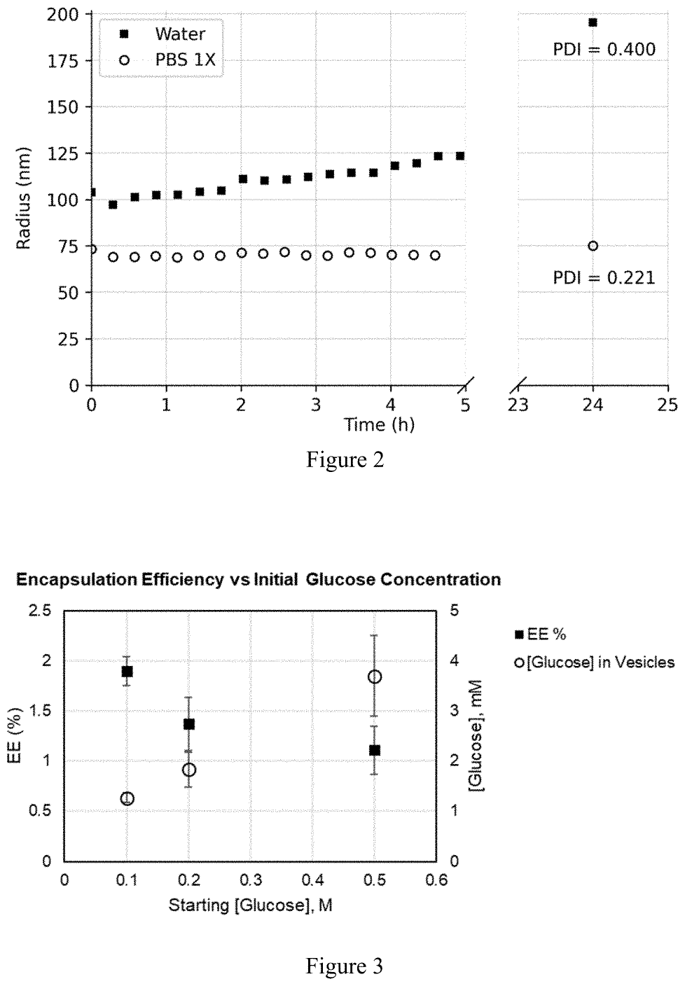

Embodiments according to the present disclosure include the use of surfactant vesicles with thermodynamic, cell-targeting, and functionalization properties that indicate their use in research, diagnostic, and therapeutic applications. The word “liposome” is used to refer to conventional vesicle formulations in which the major components are phospholipids. The word “vesicle” or “catanionic vesicle” is used to refer to spontaneously formed unilamellar bilayers in which the primary major components are two oppositely charged single-tailed surfactants enclosing an inner water pool (lumen). FIG. 1 presents a cartoon of the surfactant vesicle system described in the present disclosure. Catanionic surfactant vesicles (SVs) can be spontaneously generated when a mixture of cationic and anionic surfactants are combined with water under appropriate proportions. Vesicle formation under such conditions is spontaneous and fairly rapid (<12 h) and yields vesicles that are thermodynamically stable. These surfactant vesicles can be stable for long periods. By contrast, phospholipid liposomes formed by sonication or extrusion through membranes are essentially kinetically-trapped, nonequilibrium structures, that tend to fuse or rupture to form lamellar phases, in the process, releasing their contents.

The present disclosure provides catanionic surfactant vesicles (SVs), which may simply be referred to as “vesicles” throughout the present disclosure. The vesicles may be functionalized on their outer leaflet such that they may be recognized by cell surface receptors on cells and are thus biologically active. The vesicles may encapsulate (at least partially in the lumen and/or at least partially in the leaflet) one or more small molecules, one or more RNAs, one or more DNAs, and/or one or more proteins/peptides. Also provided are compositions comprising the vesicles (e.g., vaccine compositions comprising the vesicles) and methods of making and using the same.

In an aspect, the present disclosure provides catanionic surfactant vesicles. The vesicles comprise a plurality of surfactants. The surfactants comprise a plurality of cationic surfactants and anionic surfactants. The cationic surfactants and anionic surfactants define a leaflet having an inner leaflet surface and an outer leaflet surface and the inner leaflet defines a lumen.

The size and curvature properties (shape) of catanionic vesicles of the present disclosure can vary depending upon factors such as the length of the hydrocarbon tail regions of the constituent surfactants and the nature of the polar head groups. In various embodiments, groups functionalized on the outer leaflet of the vesicle have no observable effect on vesicle shape, size, or stability in aqueous media (e.g., at a concentration of 1:100 surface group to total surfactant ratio). The diameter of vesicles according to the invention can be, for example from about 100 to about 250 nanometers, including all nm values and ranges therebetween (e.g., about 50 to about 150 nm). The vesicle size can be influenced by selecting the relative lengths of the hydrocarbon tail regions of the anionic and cationic surfactants. For example, large vesicles, e.g., vesicles of from 150 to 200 nanometers diameter, can be formed when there is disparity between the length of the hydrocarbon tail on the anionic surfactant and the hydrocarbon tail on the cationic surfactant. For example, large vesicles can be formed when a C₁₆ cationic surfactant solution is combined with a C₈ anionic surfactant solution. Smaller vesicles can be produced by using anionic and cationic surfactant species of which the lengths of the hydrocarbon tails are more closely matched. The permeability characteristics of vesicles according to the present invention can be influenced by the nature of the constituent surfactants, for example, the chain length of the hydrocarbon tail regions of the surfactants. Longer tail lengths on the surfactant molecules can decrease the permeability of the vesicles by increasing the thickness and hydrophobicity of the vesicle membrane (leaflet).

The vesicles of the present disclosure comprise a mixture of cationic surfactants and anionic surfactants. The surfactants can be single-tailed monoalkyl surfactants. As is known in the art, surfactants in general are a broad class of structurally diverse molecules. Surfactants are amphipathic molecules composed of one or more than one hydrophobic hydrocarbon region referred to as the “tail” region, and a hydrophilic, polar region referred to as the “head region” or “head group.” The amphipathic nature of these molecules governs their behavior at and influence upon phase interfaces. Surfactants that can be used to form catanionic vesicles according to the present invention include, for example, SDS, DTAC, DTAB, DPC, DDAO, DDAB, SOS, and AOT. Exemplary anionic, single-chain surface active agents include alkyl sulfates, alkyl sulfonates, alkyl benzene sulfonates, and saturated or unsaturated fatty acids and their salts. Moieties comprising the polar head group in the cationic surfactant can include, for example, quaternary ammonium, pyridinium, sulfonium, and/or phosphonium groups. For example, the polar head group can include trimethylammonium. Exemplary cationic, single-chain surface active agents include alkyl trimethylammonium halides, alkyl trimethylammonium tosylates, and N-alkyl pyridinium halides.

Alkyl sulfates can include sodium octyl sulfate, sodium decyl sulfate, sodium dodecyl sulfate, and sodium tetra-decyl sulfate. Alkyl sulfonates can include sodium octyl sulfonate, sodium decyl sulfonate, and sodium dodecyl sulfonate. Alkyl benzene sulfonates can include sodium octyl benzene sulfonate, sodium decyl benzene sulfonate, and sodium dodecyl benzene sulfonate. Fatty acid salts can include sodium octanoate, sodium decanoate, sodium dodecanoate, and the sodium salt of oleic acid.

Alkyl trimethylammonium halides can include octyl trimethylammonium bromide, decyl trimethylammonium bromide, dodecyl trimethylammonium bromide, myristyl trimethylammonium bromide, and cetyl trimethylammonium bromide. Alkyl trimethylammonium tosylates can include octyl trimethylammonium tosylate, decyl trimethylammonium tosylate, dodecyl trimethylammonium tosylate, myristyl trimethylammonium tosylate, and cetyl trimethylammonium tosylate. For example, N-alkyl pyridinium halides can include decyl pyridinium chloride, dodecyl pyridinium chloride, cetyl pyridinium chloride, decyl pyridinium bromide, dodecyl pyridinium bromide, cetyl pyridinium bromide, decyl pyridinium iodide, dodecyl pyridinium iodide, cetyl pyridinium iodide.

In various embodiments, the cationic surfactant is cetyltrimethylammonium tosylate (CTAT) and the anionic surfactant is sodium dodecylbenzene sulfonate (SDBS). The vesicles may comprise the surfactants in various ratios. For example, the ratio of SDBS to CTAT (w/w) is 60:40 to 80:20, including all ratio values and ranges therebetween (e.g., 60:40, 61:39, 62:38, 63:37, 64:36, 65:35, 66:34: 67:33, 68:32, 69:31, 70:30, 71:29, 72:28, 73:27, 74:26, 75:25, 76:24, 77:23, 78:22, 79:21, or 80:20). In various embodiments, the ratio of SDBS to CTAT (w/w) is 65:35 to 70:30. In various embodiments, the ratio of SDBS to CTAT (w/w) is 65:35. In various other embodiments, the ratio of SDBS to CTAT (w/w) is 70:30.

As described herein, the outer leaflet of the vesicle may be functionalized with various groups. Non-limiting examples of groups include with one or more conjugation groups, one or more cell surface receptor binders, one or more small molecules, one or more peptides and/or proteins, one or more carbohydrates, one or more glycans, one or more polysaccharides, one or more nucleic acid derivatives, one or more lipids, and/or one or more monoclonal antibodies. The groups displayed on the surface of the outer leaflet may be referred to as bioconjugates.

The presented bioconjugate can interact with natural or artificial carbohydrate and/or protein recognition systems. These carbohydrate- and/or protein-functionalized vesicles present binding residues to an actual cell surface and may facilitate multivalent interactions.

A glycoconjugate can include a carbohydrate that is covalently linked to another chemical species. Examples of glycoconjugates include glycoproteins, glycopeptides, peptidoglycans, glycolipids, lipopolysaccharides, and carbohydrates covalently linked to one or more alkyl chains.

A carbohydrate or saccharide can include monosaccharides, oligosaccharides, and polysaccharides. An oligosaccharide can be formed of a few covalently linked, and a polysaccharide can be formed of many covalently linked monosaccharide units. A monosaccharide can be formed of an aldehyde or ketone with attached hydroxyl groups. Examples of monosaccharides include aldohexoses, such as glucose, aldopentoses, such as ribose, and ketohexoses, such as fructose. Monosaccharides can exist in a straight-chain or in a cyclic form, e.g., a furanose or pyranose. Carbohydrates can be displayed on the outer surface of the membranes of cells. For example, carbohydrates displayed in antigens on the surface of erythrocytes or red blood cells are responsible for the blood type of an animal.

The carbohydrate and/or peptide moiety of a bioconjugate can be selected to bind with a receptor on a target cell or another target structure. For example, the carbohydrate moiety can be selected to bind with a carbohydrate receptor on a lectin, for example, a lectin that is free in a solution or a lectin that is displayed on the outer surface of the membrane of a cell.

Lectins include proteins that have binding sites for carbohydrate moieties. For example, lectins can play a role in the immune response of an organism by binding to carbohydrates displayed on the surface of pathogens such as bacteria, parasites, yeasts, and viruses. For example, lectins can play a role in the attachment of bacteria to host cells.

Various groups that bind to cell surface receptors are suitable for display on the outer leaflet, which may be referred to as cell surface receptor binders. For example, there may be several different cell surface receptor binders displayed on the outer leaflet (e.g., at least a portion of cell surface receptor binders are different than another portion of cell surface receptor binders). In various examples, all the cell surface receptor binders are the same. Non-limiting examples of cell receptors are folate receptors. In various examples, there may be about 50 or less cell surface receptor binders displayed on the outer leaflet (e.g., 49 or less, 48 or less, 47 or less, 46 or less, 45 or less, 44 or less, 43 or less, 42 or less, 41 or less, 40 or less, 39 or less, 38 or less, 37 or less, 36 or less, 35 or less, 34 or less, 33 or less, 32 or less, 31 or less, 30 or less, 29 or less, 28 or less, 27 or less, 26 or less, 25 or less, 24 or less, 23 or less, 22 or less, 21 or less, 20 or less, 19 or less, 18 or less, 17 or less, 16 or less, 15 or less, 14 or less, 13 or less, 12 or less, 11 or less, 10 or less, 9 or less, 8 or less, 7 or less, 6 or less, or 5 or less). In various examples, there are 5 or more cell surface receptor binders displayed on the outer leaflet (e.g., 5 or more, 10 or more, 15 or more, 20 or more, 25 or more, 30 or more, 35 or more, 40 or more, 45 or more, or at least 50).

Various monoclonal antibodies are suitable for display on the outer leaflet. For example, there may be at least two different monoclonal antibodies displayed on the outer leaflet. In various examples, all the monoclonal antibodies are the same. Non-limiting examples of monoclonal antibodies include Herceptin, Rituximab, nivolumab, and other monoclonal antibodies that bind to cell surface receptors. In various examples, one monoclonal antibody is displayed on the surface of the outer leaflet. In various examples, two monoclonal antibodies are displayed on the surface of the outer leaflet. In various examples, three monoclonal antibodies are displayed on the surface of the outer leaflet. In various examples, four monoclonal antibodies are displayed on the surface of the outer leaflet.

Various conjugation groups may be displayed on the surface of the outer leaflet. For example, the conjugation group may be a functional group upon which conjugation chemistry may be performed. For example, chemistry can be acylation chemistry, a substitution reaction, a click reaction, or the like. Other conjugation chemistries and reactions are known in the art and will be known by those skilled in the art. Non-limiting examples of conjugation groups include alkynes, azides, thiols, disulfides, maleimides, thioesters, and cyanuric chloride derivatives. For example, an alkyne may be a cyclooctynyl group.

Various small molecules may be incorporated or encapsulated by a vesicle of the present disclosure. For example, a small molecule may be incorporated (partially or completely) in the lumen or leaflet of a vesicle. In various examples, one or more small molecules in partially in the lumen and partially in the leaflet. Examples of small molecules include but are not limited to chemotherapy drugs or agents, saccharides, antibiotics, or any other molecule with biological activity. In various examples, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, or at least 500 small molecules are encapsulated or incorporated in the vesicles. In various examples, all the small molecules are the same. In various other examples, at least a portion of the small molecules are different than a portion of the other small molecules encapsulated or incorporated in the vesicles.

Various chemotherapy agents (e.g., chemotherapy drugs) can be used. Any FDA approved chemotherapy agents (e.g., chemotherapy drugs) can be used. Combinations of chemotherapy agents can be used. Non-limiting examples of chemotherapy agents and combinations include abemaciclib, abiraterone acetate, ABITREXATE® (methotrexate), ABVD (doxorubicin, bleomycin, vinblastine, and dacarbazine), ABVE (doxorubicin, bleomycin, vincristine sulfate, etoposide phosphate), ABVE-PC (doxorubicin, bleomycin, vincristine sulfate, etoposide phosphate, prednisone, cyclophosphamide), AC (doxorubicin and cyclophosphamide), acalabrutinib, AC-T (doxorubicin, cyclophosphamide, paclitaxel), ADE (cytarabine, daunorubicin, etoposide), ADRIAMYCIN® (doxorubicin hydrochloride), afatinib dimaleate, AFINITOR® (everolimus), AKYNZEO® (netupitant and palonosetron hydrochloride), ALDARA® (imiquimod), aldesleukin, ALECENSA® (alectinib), alectinib, ALIMTA® (pemetrexed disodium), ALIQOPA® (copanlisib hydrochloride), ALKERAN® for injection (melphalan hydrochloride), ALKERAN® tablets (melphalan), ALOXI® (palonosetron hydrochloride), ALUNBRIG™ (brigatinib), ambochlorin (chlorambucil), amboclorin (chlorambucil), amifostine, aminolevulinic acid, anastrozole, aprepitant, AREDIA® (pamidronate disodium), ARIMIDEX® (anastrozole), AROMASIN® (exemestane), ARRANON® (nelarabine), arsenic trioxide, asparaginase Erwinia chrysanthemi, axicabtagene ciloleucel, axitinib, azacitidine, BEACOPP (bleomycin, etoposide, doxorubicin, cyclophosphamide, vincristine, procarbazine, prednisone), Becenum® (carmustine), Beleodaq® (belinostat), belinostat, bendamustine hydrochloride, BEP (bleomycin, etoposide, cisplatin), bexarotene, bicalutamide, BICNU® (carmustine), bleomycin, bortezomib, Bosulif® (bosutinib), bosutinib, brigatinib, BuMel (busulfan, melphalan hydrochloride), busulfan, BUSULFEX® (busulfan), cabazitaxel, CABOMETYX™ (cabozantinib-S-malate), cabozantinib-S-malate, CAF (cyclophosphamide, doxorubicin, 5-fluorouracil), CALQUENCE® (acalabrutinib), CAMPTOSAR® (irinotecan hydrochloride), capecitabine, CAPDX, CARAC™ (fluorouracil—topical), carboplatin, carboplatin—TAXOL®, carfilzomib, carmubris (carmustine), carmustine, carmustine implant, CASODEX® (bicalutamide), CEM (carboplatin, etoposide, melphalan), ceritinib, CERUBIDINE® (daunorubicin hydrochloride), CEV (carboplatin, etoposide phosphate, vincristine sulfate), chlorambucil, chlorambucil-prednisone, CHOP (cyclophosphamide, doxorubicin, vincristine, prednisone), cisplatin, cladribine, CLAFEN® (cyclophosphamide), clofarabine, CLOFAREX® (clofarabine), CLOLAR® (clofarabine), CMF (cyclophosphamide, methotrexate, fluorouracil), cobimetinib, COMETRIQ® (cabozantinib-S-malate), copanlisib hydrochloride, COPDAC (cyclophosphamide, vincristine sulfate, prednisone, dacarbazine), COPP (cyclophosphamide, vincristine, procarbazine, prednisone), COPP-ABV (cyclophosphamide, vincristine, procarbazine, prednisone, doxorubicin, bleomycin, vinblastine sulfate), COSMEGEN® (dactinomycin), COTELLIC® (cobimetinib), crizotinib, CVP (cyclophosphamide, vincristine, prednisolone), cyclophosphamide, CYFOS® (ifosfamide), cytarabine, cytarabine liposome, CYTOSAR-U® (cytarabine), CYTOXAN® (cyclophosphamide), dabrafenib, dacarbazine, DACOGEN® (decitabine), dactinomycin, dasatinib, daunorubicin hydrochloride, daunorubicin hydrochloride and cytarabine liposome, decitabine, defibrotide sodium, DEFITELIO® (defibrotide sodium), degarelix, denileukin diftitox, dexamethasone, dexrazoxane hydrochloride, docetaxel, doxorubicin, doxorubicin hydrochloride, doxorubicin hydrochloride liposome, DOX-SL® (doxorubicin hydrochloride liposome), DTIC-DOME® (dacarbazine), ELITEK® (rasburicase), ELLENCE® (epirubicin hydrochloride), ELOXATIN® (oxaliplatin), eltrombopag olamine, EMEND® (aprepitant), enasidenib mesylate, enzalutamide, epirubicin hydrochloride, EPOCH (etoposide, prednisone, vincristine, cyclophosphamide, and doxorubicin hydrochloride), eribulin mesylate, ERIVEDGE® (vismodegib), erlotinib hydrochloride, ERWINAZE® (asparaginase Erwinia chrysanthemi), ETHYOL® (amifostine), ETOPOPHOS® (etoposide phosphate), etoposide, etoposide phosphate, everolimus, EVISTA® (raloxifene hydrochloride), EVOMELA® (melphalan hydrochloride), exemestane, 5-FU (fluorouracil), FARESTON® (toremifene), FARYDAK® (panobinostat), FASLODEX® (fulvestrant), FEC (5-fluorouracil, epirubicin, cyclophosphamide), FEMARA® (letrozole), filgrastim, FLUDARA® (fludarabine phosphate), fludarabine phosphate, flutamide, FOLEX® (methotrexate), FOLEX PFS® (methotrexate), FOLFIRI (leucovorin calcium, fluorouracil, irinotecan hydrochloride), FOLFIRINOX (leucovorin calcium, fluorouracil, irinotecan hydrochloride, oxaliplatin), FOLFOX (leucovorin calcium, fluorouracil, oxaliplatin), FOLOTYN® (pralatrexate), FU-LV (fluorouracil, leucovorin calcium), fulvestrant, gefitinib, gemcitabine hydrochloride, gemcitabine-cisplatin, gemcitabine-oxaliplatin, GEMZAR® (gemcitabine hydrochloride), GILOTRIF® (afatinib dimaleate), GLEEVEC® (imatinib mesylate), GLIADEL® (carmustine implant), goserelin acetate, HALAVEN® (eribulin mesylate), HEMANGEOL® (propranolol hydrochloride), Hycamtin® (topotecan hydrochloride), HYDREA® (hydroxyurea), hydroxyurea, Hyper-CVAD (course A: cyclophosphamide, vincristine, doxorubicin, dexamethasone, cytarabine, mesna, methotrexate; and course B: methotrexate, leucovorin, sodium bicarbonate, cytarabine), IBRANCE® (palbociclib), ibrutinib, ICE (ifosfamide, mesna, carboplatin, etoposide), ICLUSIG® (ponatinib hydrochloride), IDAMYCIN® (idarubicin hydrochloride), idarubicin hydrochloride, idelalisib, IDHIFA® (enasidenib mesylate), IFEX® (ifosfamide), ifosfamide, IFOSFAMIDUM™ (ifosfamide), imatinib mesylate, IMBRUVICA® (ibrutinib), imiquimod, IMLYGIC® (talimogene laherparepvec), INLYTA® (axitinib), IRESSA® (gefitinib), irinotecan, irinotecan hydrochloride, irinotecan hydrochloride liposome, ISTODAX® (romidepsin), ixabepilone, ixazomib citrate, IXEMPRA® (ixabepilone), JAKAFI® (ruxolitinib phosphate), JEB (carboplatin, etoposide phosphate, bleomycin), JEVTANA® (cabazitaxel), KEOXIFENE™ (raloxifene hydrochloride), KEPIVANCE® (palifermin), KISQALI® (ribociclib), KYMRIAH™ (tisagenlecleucel), KYPROLIS® (carfilzomib), lanreotide acetate, lapatinib ditosylate, lenalidomide, lenvatinib mesylate, LENVIMA® (lenvatinib mesylate), letrozole, leucovorin calcium, LEUKERAN® (chlorambucil), leuprolide acetate, LEUSTATIN® (cladribine), LEVULAN® (aminolevulinic acid), LINFOLIZIN™ (chlorambucil), lomustine, LONSURF® (trifluridine and tipiracil hydrochloride), LUPRON® (leuprolide acetate), LUPRON DEPOT® (leuprolide acetate), LUPRON DEPOT-PED® (leuprolide acetate), LYNPARZA® (olaparib), MATULANE® (procarbazine hydrochloride), mechlorethamine hydrochloride, megestrol acetate, MEKINIST® (trametinib), melphalan, melphalan hydrochloride, mercaptopurine, mesna, MESNEX® (Mesna), METHAZOLASTONE™ (temozolomide), methotrexate, METHOTREXATE LPF™ (methotrexate), methylnaltrexone bromide, MEXATE® (methotrexate), MEXATE-AQ™ (methotrexate), midostaurin, mitomycin C, mitoxantrone hydrochloride, MITOZYTREX™ (mitomycin C), MOPP (mustargen, vincristine, procarbazine, prednisone), MOZOBIL™ (plerixafor), MUSTARGEN® (mechlorethamine hydrochloride), MUTAMYCIN™ (mitomycin C), MYLERAN® (busulfan), MYLOSAR® (azacitidine), NAVELBINE® (vinorelbine tartrate), nelarabine, NEOSAR® (cyclophosphamide), neratinib maleate, NERLYNX® (neratinib maleate), netupitant and palonosetron hydrochloride, NEULASTA® (pegfilgrastim), NEUPOGEN® (filgrastim), NEXAVAR® (sorafenib tosylate), NILANDRON® (nilutamide), nilotinib, nilutamide, NINLARO® (ixazomib citrate), niraparib tosylate monohydrate, NOLVADEX® (tamoxifen citrate), NPLATE® (romiplostim), ODOMZO® (sonidegib), OEPA (vincristine sulfate, etoposide phosphate, prednisone, doxorubicin hydrochloride), OFF (oxaliplatin, fluorouracil, leucovorin), olaparib, omacetaxine mepesuccinate, ondansetron hydrochloride, ONTAK® (denileukin diftitox), OPPA (vincristine sulfate, procarbazine hydrochloride, prednisone, doxorubicin hydrochloride), osimertinib, oxaliplatin, paclitaxel, PAD (bortezomib, doxorubicin hydrochloride, dexamethasone), palbociclib, palifermin, palonosetron hydrochloride, pamidronate disodium, panobinostat, paraplat (carboplatin), PARAPLATIN® (carboplatin), pazopanib hydrochloride, PCV (procarbazine hydrochloride, lomustine, vincristine sulfate), PEB (cisplatin, etoposide phosphate, bleomycin), pegfilgrastim, pemetrexed disodium, PLATINOL® (cisplatin), PLATINOL®-AQ (cisplatin), plerixafor, pomalidomide, POMALYST® (pomalidomide), ponatinib hydrochloride, pralatrexate, prednisone, procarbazine hydrochloride, PROMACTA® (eltrombopag olamine), propranolol hydrochloride, PURINETHOL® (mercaptopurine), PURIXAN® (mercaptopurine), radium 223 dichloride, raloxifene hydrochloride, rasburicase, regorafenib, RELISTOR® (methylnaltrexone bromide), REVLIMID® (lenalidomide), RHEUMATREX® (methotrexate), ribociclib, rolapitant hydrochloride, romidepsin, romiplostim, rubidomycin (daunorubicin hydrochloride), RUBRACA® (rucaparib camsylate), rucaparib camsylate, ruxolitinib phosphate, RYDAPT® (midostaurin), SCLEROSOL® Intrapleural Aerosol (Talc), sipuleucel-T, SOMATULINE® Depot (lanreotide acetate), sonidegib, sorafenib tosylate, SPRYCEL® (dasatinib), Stanford V (mechlorethamine hydrochloride, doxorubicin hydrochloride, vinblastine sulfate, vincristine sulfate, bleomycin, etoposide phosphate, prednisone), sterile talc powder (Talc), STERITALC® (Talc), STIVARGA® (regorafenib), sunitinib malate, SUTENT® (sunitinib malate), SYNRIBO™ (omacetaxine mepesuccinate), TABLOID® (thioguanine), TAC (docetaxel, doxorubicin hydrochloride, cyclophosphamide), TAFINLAR® (dabrafenib), TAGRISSO® (osimertinib), Talc, tamoxifen citrate, TARABINE PFS® (cytarabine), TARCEVA® (erlotinib hydrochloride), TARGRETIN® (bexarotene), TASIGNA® (nilotinib), TAXOL® (Paclitaxel), TAXOTERE® (docetaxel), TEMODAR® (temozolomide), temozolomide, temsirolimus, thalidomide, THALOMID® (thalidomide), thioguanine, thiotepa, TOTECT® (dexrazoxane hydrochloride), TPF (docetaxel, cisplatin, fluorouracil), trabectedin, trametinib, TREANDA® (bendamustine hydrochloride), trifluridine and tipiracil hydrochloride, TRISENOX® (arsenic trioxide), TYKERB® (lapatinib ditosylate), uridine triacetate, VAC (vincristine sulfate, dactinomycin, cyclophosphamide), valrubicin, VALSTAR® (valrubicin), vandetanib, VAMP (vincristine sulfate, doxorubicin hydrochloride, methotrexate, prednisone), VARUBI® (rolapitant hydrochloride), VeIP (vinblastine sulfate, ifosfamide, cisplatin), VELBAN® (vinblastine sulfate), VELCADE® (bortezomib), VELSAR® (vinblastine sulfate), vemurafenib, VENCLEXTA™ (venetoclax), venetoclax, VERZENIO™ (abemaciclib), VIADUR® (leuprolide acetate), VIDAZA® (azacitidine), vinblastine sulfate, VINCASAR PFS® (vincristine sulfate), vincristine sulfate, vinorelbine tartrate, VIP (etoposide phosphate, ifosfamide, cisplatin), vismodegib, VISTOGARD® (uridine triacetate), vorinostat, VOTRIENT® (pazopanib hydrochloride), WELLCOVORIN® (leucovorin calcium), XALKORI® (crizotinib), XELODA® (capecitabine), XELIRI (capecitabine, irinotecan hydrochloride), XELOX (capecitabine, oxaliplatin), XOFIGO® (radium 223 dichloride), XTANDI® (enzalutamide), YESCARTA™ (axicabtagene ciloleucel), YONDELIS® (trabectedin), ZALTRAP® (ziv-aflibercept), ZARXIO® (filgrastim), ZEJULA® (niraparib tosylate monohydrate), ZELBORAF® (vemurafenib), ZINECARD® (dexrazoxane hydrochloride), ZOFRAN® (ondansetron hydrochloride), ZOLADEX® (goserelin acetate), zoledronic acid, ZOLINZA® (vorinostat), ZOMETA® (zoledronic acid), ZYDELIG® (idelalisib), ZYKADIA® (ceritinib), and ZYTIGA® (abiraterone acetate).

In various examples, the chemotherapy drug or agent is doxorubicin, cisplatin, carboplatin, pemetrexed, auristatin, maytansine, paclitaxel, camptothecin, vincristine, vinblastine, irinotecan, amphotericin B, salts thereof, or combinations thereof.

In various embodiments, a vesicle of the present disclosure encapsulated or incorporates one or more proteins/peptides, one or more RNAs, and/or one or more DNAs. In various embodiments, the vesicles incorporate one or more siRNAs. In various examples, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, or at least 500 siRNAs are encapsulated or incorporated in the vesicles. In various examples, all the siRNAs are the same. In various other examples, at least a portion of the RNAs are different than a portion of the other siRNAs encapsulated or incorporated in the vesicles.

Various DNAs and/or RNAs may be encapsulated or incorporated into the vesicles of the present disclosure. When incorporating or encapsulating DNAs and/or RNAs, the vesicles may further comprise one or more polyamines and/or condensed polyamines. For example, the polyamine may be spermine. The DNA and/or RNA of various lengths and sizes may be incorporated or encapsulated by a vesicle of the present disclosure. For example, the DNA and/or RNA have 20 or more nucleobases or nucleobase pairs (e.g., 20-15,000 nucleobases or nucleobase pairs (including all integer values and ranges therebetween), 200-300 nucleobases or nucleobase pairs, 500-10,000 nucleobases or nucleobase pairs, or 10,000-15,000 nucleobases or nucleobase pairs). In various embodiments, the one or more RNAs are amino acid-encoding RNAs, such as, for example, mRNA. The RNAs may have various elements such as internal ribosome entry site (IRES) elements. In various embodiments, the one or more DNAs are plasmids. For example, the plasmids may be plasmids for genes derived from viral pathogens including but not limited to SARS Covid-2, polio, smallpox, monkeypox, hemorrhagic viruses such as SARS, MERS, Ebola, Marburg virus, influenza, Human Papilloma virus, measles, mumps, Rubella, Herpes, Shingles (Chickenpox), Shigella, chikungunya virus, Dengue, diphtheria, meningitis, and the like.

Various proteins/peptides may be incorporated or encapsulated by vesicles of the present disclosure. Proteins/peptides of various lengths, sizes, secondary structures, tertiary structures, and quaternary structures may be incorporated or encapsulated by the vesicles. For example, any protein with hydrodynamic radius of about ⅔ of lumen volume could be incorporated into the vesicle, including, but not limited to, Spike protein, diphtheria toxin, Staphylococcus aureus toxin, and the like, and combinations thereof.

In an aspect, the present disclosure provides a composition. The composition comprises a vesicle of the present disclosure.

A composition can comprise one or more vesicles in a pharmaceutically acceptable carrier (e.g., carrier). The carrier can be an aqueous carrier suitable for administration to individuals including humans. The carrier can be sterile. The carrier can be a physiological buffer. Examples of suitable carriers include sucrose, dextrose, saline, and/or a pH buffering element (such as, a buffering element that buffers to, for example, a pH from pH 5 to 9, from pH 6 to 8, (e.g., 6.5)) such as histidine, citrate, or phosphate. Additionally, pharmaceutically acceptable carriers may be determined in part by the particular composition being administered, as well as by the particular method used to administer the composition. Accordingly, there are a wide variety of suitable formulations of pharmaceutical compositions of the present disclosure. Additional, non-limiting examples of carriers include solutions, suspensions, emulsions, solid injectable compositions that are dissolved or suspended in a solvent before use, and the like. Injections may be prepared by dissolving, suspending, or emulsifying one or more of active ingredients in a diluent. Examples of diluents, include, but are not limited to distilled water for injection, physiological saline, vegetable oil, alcohol, dimethyl sulfoxide, and the like, and combinations thereof. Compositions may contain stabilizers, solubilizers, suspending agents, emulsifiers, soothing agents, buffers, preservatives, and the like, and combinations thereof. Compositions may be sterilized or prepared by sterile procedure. A composition of the disclosure may also be formulated into a sterile solid preparation, for example, by freeze-drying, and may be used after sterilization or dissolution in sterile injectable water or other sterile diluent(s) immediately before use. Additional examples of pharmaceutically acceptable carriers include, but are not limited to, sugars, such as, for example, lactose, glucose, and sucrose; starches, such as, for example, corn starch and potato starch; cellulose, including sodium carboxymethyl cellulose, ethyl cellulose, and cellulose acetate; powdered tragacanth; malt; gelatin; talc; excipients, such as cocoa butter and suppository waxes; oils, such as, for example, peanut oil, cottonseed oil, safflower oil, sesame oil, olive oil, corn oil, and soybean oil; glycols, such as, for example, propylene glycol; polyols, such as, for example glycerin, sorbitol, mannitol, and polyethylene glycol; esters, such as, for example, ethyl oleate and ethyl laurate; agar; buffering agents, such as, for example, magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Additional non-limiting examples of pharmaceutically acceptable carriers can be found in: Remington: The Science and Practice of Pharmacy (2012) 22nd Edition, Philadelphia, Pa. Lippincott Williams & Wilkins. Parenteral administration may be prepared and include infusions such as, for example, intramuscular, intravenous, intraarterial, intraperitoneal, subcutaneous administration, and the like. For example, composition comprises vesicles of the present disclosure and a sterile, suitable carrier for administration to individuals including humans—such as a physiological buffer such as sucrose, dextrose, saline, pH buffering (such as from pH 5 to 9, from pH 7 to 8, from pH 7.2 to 7.6, (e.g., 7.4)) element such as histidine, citrate, or phosphate.

The pharmaceutical compositions or formulations of the application, in addition to comprising a vesicle, may further comprising one or more of the following excipients: antioxidant, buffering agent, bulking agent, non-aggregating agent, binding agent, filler, diluent (e.g., starches or partially gelatinized starches, sorbitol, mannitol, maltitol, microcrystalline cellulose); disintegrant (e.g., sodium croscarmellose and sodium starch glycolate); plasticizers (e.g., glycerol, vitamin E TPGS, triacetin); anti-tacking agent (e.g., tricalcium phosphate, silicon dioxide, bentonite); wetting agent (sodium lauryl sulfate, sodium stearyl fumarate, polyoxyethylene 20 sorbitan mono-oleate (e.g., Tween™); sweetener (sucralose, sorbitol, and xylitol); colorant (FD&C Blue #1 Aluminum Lake, FD&C Blue #2, other FD&C Blue colors, titanium dioxide, iron oxide); flavorant (menthol, peppermint oil, almond oil); glidant (colloidal silica, precipitated silica, and talc); pH adjuster (arginine, tartaric acid, sodium hydrogen carbonate, adipic acid); or surfactant (ammonium lauryl sulfate, sodium lauryl sulfate (sodium dodecyl sulfate, SLS, or SDS), sodium laureth sulfate, sodium myreth sulfate, dioctyl sodium sulfosuccinate, fatty acid esters of glycerol, poloxamers).

Antioxidants, include, without limitation, hindered phenols (e.g., tetrakis [methylene (3,5-di-t-butyl-4-hydroxyhydrocinnamate)]methane), less-hindered phenols, and semi-hindered phenols; phosphates, phosphites, and phosphonites (e.g., tris (2,4-di-t-butylphenyl) phosphate); thio compounds (e.g., distearyl thiodipropionate, dilaurylthiodipropionate); various siloxanes; and various amines (e.g., polymerized 2,2,4-trimethyl-1,2-dihydroquinoline). In one embodiment, the antioxidant is selected from the group consisting of distearyl thiodipropionate, dilauryl thiodipropionate, octadecyl-3,5-di-t-butyl-4-hydroxyhydrocinnamate, benzenepropanoic acid, 3,5-bis (1,1-dimethylethyl)-4-hydroxy-thiodi-2,1-ehtanediyl ester, stearyl 3-(3,5-di-t-butyl-4-hydroxyphenyl) propionate, octadecyl-3-(3,5-di-tert-butyl-4-hydroxyphenyl)-propionate, 2,4-bis(dodecylthiomethyl)-6-methylphenol, 4,4′-thiobis(6-tert-butyl-m-cresol), 4,6-bis (octylthiomethyl)-o-cresol, 1,3,5-tris(4-tert-butyl-3-hydroxy-2,6-dimethyl benzyl)-1,3,5-triazine-2,4,6-(1H,3H,5H)-trione, pentaerythritol tetrakis (3-(3,5-di-t-butyl-4-hydroxyphenyl)propionate), 2′,3-bis[[3-[3,5-di-tert-butyl-4-hydroxyphenyl]propionyl]]propionohydrazide, and mixtures thereof. In one embodiment, the antioxidant is butylated hydroxyanisole, butylated hydroxytoluene (BHT), sodium metabisulfite, propyl gallate, cysteine, methionine, or ethylenediaminetetraacetic acid (EDTA).

Buffering agents are included in the pharmaceutical formulations of the application to prevent or reduce a pH change in the dosage form after administration to a subject. Representative buffering agents include, without limitation, borates, borate-polyol complexes, succinate, phosphate buffering agents, citrate buffering agents, acetate buffering agents, carbonate buffering agents, organic buffering agents, amino acid buffering agents, or combinations thereof. In one embodiment, the buffering agent is citric acid, sodium phosphate, sodium citrate, sodium acetate, sodium hydroxide, acetic acid, potassium chloride, sodium chloride, sodium bicarbonate, L-arginine, a cholic acid derivative, or tris(hydroxymethyl)aminomethane (TRIS).

Bulking agents, or fillers, include, without limitation, lactose monohydrate, microcrystalline cellulose, cellulose acetate, calcium carbonate, potato starch, sucrose, and sorbitol, and dextrose.

Non-aggregating agents, or lubricants, include, without limitation, boric acid, PEG4000, PEG6000, sodium oleate, sodium benzoate, sodium acetate, sodium acetate, sodium stearate, sodium stearyl fumarate, sodium lauryl sulfate, magnesium lauryl sulfate, magnesium stearate, stearate, stearic acid, talc, hydrogenated oil, and glyceryl behenate.

Binding agents include, without limitation, acacia, gelatin, starch paste, polyvinylpyrrolidone, polyethylene glycol, glucose, carboxymethyl cellulose, and povidone.

The pharmaceutical compositions or formulations of the application can be administered via a route selected from the group consisting of route selected from the group consisting of oral administration, nasal (intranasal) administration, administration by inhalation, rectal administration, intraperitoneal injection, intravascular injection, subcutaneous injection, transcutaneous administration, and intramuscular injection.

In various embodiments, the composition is composition suitable for a vaccine. For example, the vaccine may comprise one or more adjuvant. Examples of adjuvants include attenuated lipid A derivatives such as monophosphoryl lipid A (MPLA), or synthetic derivatives such as 3-deacylated monophosphoryl lipid A, or Monophosphoryl Hexa-acyl Lipid A, 3-Deacyl. In various embodiments, the adjuvants may be monophosphoryl lipid A (MPLA), aluminum phosphate, aluminum hydroxide, alum, phosphorylated hexaacyl disaccharide (PHAD), Sigma adjuvant system (SAS), or AddaVax (Invitrogen).

The vaccine composition may comprise one or more plasmids to induce expression of a protein that induces an immune response to a virus. The immune response generated may be selected for a desired virus. For example, the virus may be SARS Covid-2, polio, smallpox, monkeypox, hemorrhagic viruses such as SARS, MERS, Ebola, Marburg virus, influenza, Human Papilloma virus, measles, mumps, Rubella, Herpes, Shingles (Chickenpox), Shigella, chikungunya virus, Dengue, diphtheria, meningitis, and the like.

In an aspect, the present disclosure provides kits. The kits may provide the vesicles of the present disclosure or components to produce a composition of the present disclosure.

The kit may be used to prepare compositions. Compositions may be prepared at a patient's bedside or by a pharmaceutical manufacturer. In the latter case, the compositions can be provided in any suitable container, such as, for example, a sealed sterile vial, ampoule, or the like, and may be further packaged (the combination of which may be referred to as a kit) to include instruction documents for use by a pharmacist, physician, other health care provider, or the like. The compositions can be provided as a liquid, or as a lyophilized or powder form that can be reconstituted if necessary when ready for use. In particular, the compositions can be provided in combination with any suitable delivery form or vehicle, examples of which include but are not limited to liquids, caplets, capsules, tablets, inhalants or aerosol, etc. The delivery devices may comprise components that facilitate release of the pharmaceutical agents over certain time periods and/or intervals, and can include compositions that enhance delivery.

In an aspect, the present disclosure provides methods of using the vesicles and compositions. The methods may be used to treat an individual having or suspected of having cancer.

Compositions of the present disclosure can be administered to any human or non-human animal in need of therapy or prophylaxis for one or more condition(s) for which the pharmaceutical agent is intended to provide a prophylactic of therapeutic benefit. Thus, the individual can be diagnosed with, suspected of having, or be at risk for developing any of a variety of conditions for which a reduction in severity would be desirable. Non-limiting examples of such conditions include cancer, including solid tumors, blood cancers (e.g., leukemia, lymphoma, myeloma, and the like). Specific examples of cancers include, but are not limited to, fibrosarcoma, myxosarcoma, liposarcoma, chondrosarcoma, osteogenic sarcoma, chordoma, angiosarcoma, endotheliosarcoma, lymphangiosarcoma, pseudomyxoma peritonei, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, head and neck cancer, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilns' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, thymoma, Waldenstrom's macroglobulinemia, heavy chain disease, and the like.

In addition, the compositions of the present disclosure can be used in connection with treating a variety of infectious diseases. It is expected that a variety of agents used to treat and/or inhibit infectious diseases caused by, for example, bacterial, protozoal, helminthic, fungal origins, viral origins, or the like can be aided by use of compositions of the present disclosure.

Various methods known to those skilled in the art can be used to introduce the compounds and/or compositions of the present disclosure to an individual. These methods include, but are not limited to, intravenous, intramuscular, intracranial, intrathecal, intradermal, subcutaneous, oral routes, and the like, and combinations thereof. The dose of the composition comprising a compound and a pharmaceutical agent will necessarily be dependent upon the needs of the individual to whom the composition is to be administered. These factors include, but are not necessarily limited to, the weight, age, sex, medical history, and nature and stage of the disease for which a therapeutic or prophylactic effect is desired. The compositions can be used in conjunction with any other conventional treatment modality designed to improve the disorder for which a desired therapeutic or prophylactic effect is intended, non-limiting examples of which include surgical interventions and radiation therapies. The compositions can be administered once, or over a series of administrations at various intervals determined using knowledge of those skill in the art, and given the benefit of the present disclosure.

Methods of the present disclosure may be used on various individuals. In various examples, an individual is a human or non-human mammal. Examples of non-human mammals include, but are not limited to, farm animals, such as, for example, cows, hogs, sheep, and the like, as well as pet or sport animals such as, for example, horses, dogs, cats, and the like. Additional non-limiting examples of individuals include, but are not limited to, rabbits, rats, mice, and the like.

The method may comprise administering a vesicle of the present disclosure to an individual in need of prevention, treatment, amelioration, or management of a disease. The vesicle may comprise one or more small molecules, siRNAs, one or more RNAs, one or more proteins/peptides, and/or one or more DNAs. In various other embodiments, the vesicles are functionalized on the outer leaflet as described herein. In various embodiments, the vesicle may comprise one or more small molecules, siRNAs, one or more RNAs, one or more proteins/peptides, and/or one or more DNAs; and the vesicles are functionalized on the outer leaflet as described herein. In various other embodiments, the vesicles may be administered as a vaccine composition to induce an immune response to a desired target. The vesicles may be administered in a therapeutically effective amount. The therapeutically effective amount may be of the cargo encapsulated or incorporated in the vesicles. The term “effective amount” as used herein refers to an amount of an agent or combination of agents (e.g., chemotherapy agent(s) and/or other agent as described herein) sufficient to achieve, in a single or multiple doses or administration(s), the intended purpose or achieve a desired result of the administration. The exact amount desired or required will vary depending on the particular compound or composition used, its mode of administration, type of cancer, patient specifics, and the like. Appropriate effective amount can be determined by one skilled in the art informed by the instant disclosure using only routine experimentation.

The steps of the method described in the various embodiments and examples disclosed herein are sufficient to carry out the methods of the present invention. Thus, in an embodiment, the method consists essentially of a combination of the steps of the methods disclosed herein. In another embodiment, the method consists of such steps.

The following Statements provide various examples of the present disclosure.

Statement 1. A catanionic surfactant vesicle comprising a cationic surfactant and an anionic surfactant, wherein the cationic surfactant is cetyltrimethylammonium tosylate (CTAT) and the anionic surfactant is sodium dodecylbenzene sulfonate (SDBS) and the SDBS and CTAT are present in a ratio of 60:40 to 80:20 (SDBS:CTAT, w/w); the cationic surfactants and the anionic surfactants define a leaflet having inner leaflet and an outer leaflet and the inner leaflet defines a lumen; and the catanionic surfactant vesicle at least partially encapsulates one or more small molecules and/or one or more siRNAs.
Statement 2. A catanionic surfactant vesicle according to Statement 1, wherein the ratio of SDBS to CTAT is 65:35 to 70:30.
Statement 3. A catanionic surfactant vesicle according to Statements 1 or 2, wherein the ratio of SDBS to CTAT is 70:30.
Statement 4. A catanionic surfactant vesicle according to Statements 1 or 2, wherein the ratio of SDBS to CTAT is 65:35.
Statement 5. A catanionic surfactant vesicle according to any one of the preceding Statements, wherein the outer leaflet of the catanionic surfactant vesicle is functionalized with one or more conjugation groups, one or more cell surface receptor binders, one or more small molecules, one or more peptides and/or proteins, one or more carbohydrates, one or more glycans, one or more polysaccharides, one or more nucleic acid derivatives, one or more lipids, and/or one or more monoclonal antibodies.
Statement 6. A catanionic surfactant vesicle according to claim Statement 5, wherein the one or more cell surface receptor binders are folate receptors.
Statement 7. A catanionic surfactant vesicle according to Statement 5, wherein the one or more monoclonal antibodies are Herceptin, Rituximab, nivolumab, or a combination thereof.
Statement 8. The catanionic surfactant vesicle according to Statement 5, wherein the one or more conjugation groups have an alkyne, an azide, a thiol, a disulfide, a maleimide, a thioester, or a cyanuril chloride derivative.
Statement 9. A catanionic surfactant vesicle according to any one of the preceding Statements, wherein the one or more small molecules is a chemotherapy drug, saccharide, antibiotic, or biologically active group.
Statement 10. A catanionic surfactant vesicle according to Statement 9, wherein the chemotherapy drug is doxorubicin, cisplatin, carboplatin, pemetrexed, auristatin, maytansine, paclitaxel, camptothecin, vincristine, vinblastine, irinotecan, amphotericin B, salts thereof, or combinations thereof.
Statement 11. A catanionic surfactant vesicle according to according to any one of the preceding Statements, wherein the cationic surfactant vesicle encapsulates at least 100 small molecules or siRNAs.
Statement 12. A catanionic surfactant vesicle according to any one of the preceding Statements, wherein the catanionic surfactant vesicles are stable at temperatures of room temperature or greater for at least one or more hours or days. In various examples, vesicles containing doxorubicin may be stable for at least one or more years. Without intending to be bound by any particular theory, it is considered a nucleotide may be stable in the vesicle for one or more months at room temperature.
Statement 13. A catanionic surfactant vesicle according to any one of the preceding Statements, wherein the one or more small molecules and/or one or more siRNAs are partially encapsulated in the lumen and/or leaflet.
Statement 14. A catanionic surfactant vesicle comprising a cationic surfactant and an anionic surfactant, wherein the cationic surfactant is cetyltrimethylammonium tosylate (CTAT) and the anionic surfactant is sodium dodecylbenzene sulfonate (SDBS) and the SDBS and CTAT are present in a ratio of 60:40 to 80:20 (SDBS:CTAT, w/w); the cationic surfactants and the anionic surfactants define a leaflet having inner leaflet and an outer leaflet and the inner leaflet defines a lumen; and the catanionic surfactant vesicle at least partially encapsulates one or more protein, one or more DNAs, and/or one or more RNAs.
Statement 15. A catanionic surfactant vesicle according to Statement 14, wherein the ratio of SDBS to CTAT is 65:35 to 70:30.
Statement 16. A catanionic surfactant vesicle according to Statements 14 or 15, wherein the ratio of SDBS to CTAT is 70:30.
Statement 17. A catanionic surfactant vesicle according to Statements 14 or 15, wherein the ratio of SDBS to CTAT is 65:35.
Statement 18. A catanionic surfactant vesicle according to any one of Statements 14-17, wherein the outer leaflet of the catanionic surfactant vesicle is functionalized with one or more conjugation groups, one or more cell surface receptor binders, one or more small molecules, one or more peptides and/or proteins, one or more carbohydrates, one or more glycans, one or more polysaccharides, one or more nucleic acid derivatives, one or more lipids, and/or one or more monoclonal antibodies.
Statement 19. A catanionic surfactant vesicle according to Statement 18, wherein the one or more cell surface receptor binders are folate receptors.
Statement 20. A catanionic surfactant vesicle according to Statement 18, wherein the one or more monoclonal antibodies are Herceptin, Rituximab, nivolumab, or a combination thereof.
Statement 21. A catanionic surfactant vesicle according to Statement 18, wherein the one or more conjugation groups have an alkyne, an azide, a thiol, a disulfide, a maleimide, a thioester, or a cyanuril chloride derivative.
Statement 22. A catanionic surfactant vesicle according to any one of Statements 14-21, further comprising polyamines and/or condensed polyamines.
Statement 23. A catanionic surfactant vesicle according to Statement 22, wherein the polyamine is spermine.
Statement 24. A catanionic surfactant vesicle according to any one of Statements 14-23, wherein the one or more DNAs and/or one or more RNAs have 20 or more nucleobases or nucleobase pairs.
Statement 25. A catanionic surfactant vesicle according to any one of Statements 14-24, wherein the one or more proteins are Spike protein, diphtheria toxin, Staphylococcus aureus toxin, and the like, and combinations thereof.
Statement 26. A catanionic surfactant vesicle according to any one of Statements 14-25, wherein the one or more RNAs are amino acid-encoding RNAs.
Statement 27. A catanionic surfactant vesicle according to any one of Statements 14-26, wherein the one or more DNAs are plasmids.
Statement 28. A composition comprising the catanionic surfactant vesicle according to any one of Statements 1-13 and a pharmaceutical carrier.
Statement 29. A composition comprising the catanionic surfactant vesicle according to any one of Statements 14-27 and a pharmaceutical carrier.
Statement 30. A vaccine composition comprising the catanionic surfactant vesicle according to any one of Statements 14-27 and a pharmaceutical carrier and, optionally, one or more adjuvants.
Statement 31. A vaccine composition according to Statement 30, wherein the one or more DNAs are plasmids.
Statement 32. A vaccine composition according to Statement 30, wherein the one or more RNAs are amino acid-encoding RNAs.
Statement 33. A vaccine composition according to Statement 31, wherein the one or more plasmids induce expression of a protein that induces an immune response to a virus.
Statement 34. A vaccine composition according to Statement 32, wherein the virus is SARS Covid-2, polio, smallpox, monkeypox, hemorrhagic viruses such as SARS, MERS, Ebola, Marburg virus, influenza, Human Papilloma virus, measles, mumps, Rubella, Herpes, Shingles (Chickenpox), Shigella, chikungunya virus, Dengue, diphtheria, meningitis, and the like.
Statement 35. A kit comprising the composition according to Statement 28 or the components to produce the composition according to Statement 28.
Statement 36. A kit comprising the composition according to Statement 29 or the components to produce the composition according to Statement 29.
Statement 37. A kit comprising the vaccine composition according to Statement 30 or the components to produce the vaccine composition according to Statement 30.
Statement 38. A method for prevention, treatment, amelioration, or management of an individual having cancer comprising administering one or more catanionic surfactant vesicles according to any one of Statements 1-13 or a composition thereof to the individual.
Statement 39. A method according to Statement 38, wherein the cancer is hepatic cancer, colon cancer, rectal cancer, breast cancer, prostate cancer, skin cancer, head and neck cancer, lung cancer, gastric cancer, mesothelioma, melanoma, lymphoma, Barrett's esophagus, synovial sarcoma, cervical cancer, endometrial ovarian cancer, Wilm's tumor, bladder cancer, leukemia, or a combination thereof.
Statement 40. A method according to Statements 38 or 39, wherein the outer leaflet of the one or more catanionic surfactant vesicles is functionalized with one or more conjugation groups, one or more cell surface receptor binders, one or more small molecules, one or more peptides and/or proteins, one or more carbohydrates, one or more glycans, one or more polysaccharides, one or more nucleic acid derivatives, one or more lipids, and/or one or more monoclonal antibodies.
Statement 41. A method for inducing an immune response in an individual comprising administering the vaccine composition according to any one of Statements 30-34, wherein following administration, the individual has an immune response to a virus.
Statement 42. A method according to claim 41, wherein the virus is SARS Covid-2, polio, smallpox, monkeypox, hemorrhagic viruses such as SARS, MERS, Ebola, Marburg virus, influenza, Human Papilloma virus, measles, mumps, Rubella, Herpes, Shingles (Chickenpox), Shigella, chikungunya virus, Dengue, diphtheria, meningitis, or the like.
Statement 43. A method according to Statements 40-42, wherein the vaccine composition comprises one or more adjuvants.
Statement 44. A method according to Statements 40-43, wherein the outer leaflet of the one or more catanionic surfactant vesicles is functionalized with one or more conjugation groups, one or more cell surface receptor binders, one or more small molecules, one or more peptides and/or proteins, one or more carbohydrates, one or more glycans, one or more polysaccharides, one or more nucleic acid derivatives, one or more lipids, and/or one or more monoclonal antibodies.

The following examples are presented to illustrate the present disclosure. They are not intended to be limiting in any matter.

### Example 1

This example provides a description of catanionic surfactant vesicles of the present disclosure and methods of making same.

Catanionic surfactant vesicles (SVs) are a lipid-derived nanoparticle scaffold that are able to incorporate a drug payload and be decorated on the surface with cell targeting ligands. Their ease of synthesis and stability make them an attractive alternative to other vesicular drug delivery systems that have been developed for drug delivery. Described herein is the synthesis of anion rich catanionic surfactant vesicles comprising sodium dodecylbenzenesulfonate (SDBS) and cetyltrimethylammonium tosylate (CTAT) in a variety of biologically relevant cell growth media and buffers. The vesicle membrane release kinetics were measure with respect to a series of neutral and charged monosaccharides, neutral disaccharides, and maltotriose in phosphate buffered saline (PBS 1×) at pH 7.4. Finally, the anion rich SDBS/CTAT vesicles were loaded with four chemotherapeutic drugs: doxorubicin, pemetrexed, carboplatin, and cisplatin, and measured their release characteristics.

As diagrammed in FIG. 1, catanionic SVs provide three distinct environments for functionalization in drug delivery. The lumen (internal volume) of the vesicle is an aqueous environment capable of containing water-soluble molecules. The hydrophobic leaflet can be employed to incorporate hydrophobic chemotherapeutics that typically are insoluble in aqueous media. Finally, the outer leaflet of the vesicle is an aqueous environment that can be decorated with amphiphilic molecules bearing a hydrophobic chain that would lock a water-soluble targeting agent into the leaflet.

To establish trends in release rates, and to compare catanionic SVs to more extensively studied liposomal vesicles, the release rate and membrane permeability of SDBS-rich (anionic) vesicles were measured using a small library of neutral and charged monosaccharides. Anionic, SDBS-rich SVs were studied because anionic SVs are much less prone to fuse with cells; cells also have a negative charged surface, and the goal of this research is to be able to target drug delivery to specific cell populations. CTAT-rich SVs carry a positive surface charge and readily fuse with eukaryotic cells and lead to poor opportunities for targeting.

To explore the effect of increasing substrate size on membrane permeability, the kinetics of release of monosaccharides, disaccharides, as well as maltotriose were measured. The investigation of small hydrophilic sugars not only provides a grounds for comparison to liposomal membrane permeability, but the results may be extended to many of the carbohydrate-based drugs currently in production or clinical trials, including heparin. Finally, the encapsulation and kinetics of release of four common anti-cancer drugs: doxorubicin, pemetrexed, carboplatin, and cisplatin were measured.

Results and Discussion.

Anionic, SDBS-rich SVs were found to form spontaneously with a narrow size distribution and stability in a variety of biorelevant solvent mixtures as reported in Table 1. Of the mixtures analyzed, several of the buffers studied were particularly relevant for sub 0° C. storage of the formulations. Although all of these solvent systems provided SVs of approximately the same diameter (69-84 nm), the polydispersity index (PDI) of the resulting colloidal suspensions were quite different. The majority of the buffer systems provided vesicle formulations with a relatively narrow size distribution, typically ±30 nm (PDI 0.2). However, the glycerol and sucrose solutions had PDIs indicative a much larger size distributions.

TABLE 1
Average radii and poly dispersity (PDI) of anion rich
catanionic SVs in prepared in various media.

| | Vesicle Radius | |
|---|---|---|
| Medium | (nm) | PDI |
| Water | 76 | 0.21 |
| PBS 1× (pH 7.4) | 74 | 0.20 |
| McIlvaine (pH 5.0) | 69 | 0.20 |
| DMEM | 67 | 0.18 |
| Opti-MEM | 80 | 0.21 |
| McCoy's 5A | 76 | 0.20 |
| 70% Glycerol | 84 | 0.45 |
| 60% Sucrose | 81 | 0.56 |

As discussed briefly above, anionic SVs were formulated in phosphate buffered saline (PBS 1×, pH 7.4) so that the results obtained were relevant to in vivo applications such as drug delivery in eukaryotic cells. The large negative zeta (ζ)-potential (˜−50 mV) of SDBS-rich SVs imparts an exceptional stability to these lipid systems due to electrostatic repulsion between the individual vesicles. Accordingly the SVs do not fuse with themselves leading to agglutination, nor with cells in living systems because cells also possess a negative surface charge. Crude vesicle formulations were prepared as 1% w/w total surfactant solutions in order to be solidly within the vesicle forming region of the SDBS/CTAT/water ternary phase diagram, and were purified via gel filtration through Sepharose CL-2B to remove tosylate (released during vesicle formation), micelles derived from traces of unincorporated surfactants, and unencapsulated substrate.

Three different SDBS:CTAT ratios were studied initially with PBS 1× buffer to determine the most appropriate system for the incorporation and kinetic studies, and the results are summarized in Table 2. It was found that, after purification by gel filtration, 60:40 w/w vesicles prepared in PBS 1× (Table 2, A) had the highest surfactant concentration (and therefore highest vesicle number density) while retaining the same surfactant ratio as 70:30 w/w vesicles in water (Table 2, C). Vesicles formed in this manner were found to be stable indefinitely (>1 year) at room temperature in PBS 1× as evidenced by long-term DLS analysis.

TABLE 2
The ratio of surfactants in crude and purified surfactant vesicles.

| | | Crude | | | | |
|---|---|---|---|---|---|---|
| | | Vesicles | Purified Vesicles^{a} | | | |
| | | w/w % Ratio | w/w % Ratio | mol % Ratio | [SDBS], | [CTAT], |
| Entry | Medium | SDBS:CTAT | SDBS:CTAT^{b} | SDBS:CTAT^{b} | mM | mM |
| a | PBS 1X | 60:40 | 65:35 | 61:39 | 5.9 ± 0.05 | 3.7 ± 0.01 |
| b | PBS 1X | 70:30 | 71:29 | 68:32 | 5.0 ± 0.04 | 2.3 ± 0.02 |
| c | PBS 1X | 80:20 | 77:23 | 75:25 | 4.3 ± 0.06 | 1.5 ± 0.02 |
| d | Water | 60:40 | 58:42 | 54:46 | 6.9 ± 0.10 | 5.3 ± 0.01 |
| e | Water | 70:30 | 64:36 | 61:39 | 5.3 ± 0.04 | 3.5 ± 0.02 |
| f | Water | 80:20 | 64:36 | 61:39 | 3.0 ± 0.09 | 1.9 ± 0.03 |

^{a}Vesicles are purified by gel filtration as described in the Supplementary Material.
^{b}The ratio of SDBS:CTAT was determined by integration of the NMR signals at 7.50 ppm (SDBS) and 3.05 ppm (CTAT), respectively. The error is derived from three different batches of vesicles.

Encapsulation of Sugars.

Vesicles were formed in the presence of several different sugars, purified by gel filtration, and the encapsulated sugar concentration measured by NMR (Table 3). Of the neutral monosaccharides (Table 3, A-E), an encapsulation efficiency (EE) of ˜1% was measured, except for ribose which had an order of magnitude lower EE. Ribose has been shown to diffuse across fatty acid and phospholipid membranes roughly 100× faster than hexoses. Assuming ribose is similarly fast to diffuse across catanionic SV membranes, and considering release is expected to occur during purification of the vesicles through the size exclusion gel, then a decreased incorporation of ribose can be expected. The pyranoside, α-D-methyl D-glucopyranoside (Table 3, F), also gave a reduced encapsulation efficiency when compared to the pyranoses which is probably due to a larger rate of diffusion across the vesicle membrane (vide infra).

Catanionic SVs have been shown to entrap and retain substrates that possess a charge opposite that of the vesicle surface more efficiently. Accordingly, a threefold higher encapsulation efficiency was measured for glucosamine (Table 3, G) which exists primarily in the positively charged protonated form at pH 7.4. Vesicles formed in the presence of 0.5 M glucosamine were unstable, providing further evidence that there is direct interaction with the vesicle membrane (the data in Table 3 was collected for 0.1 M glucosamine solutions). On the other hand, vesicles formed in the presence of the negatively charged glucosamine-2-sulfate were stable and displayed similar EE (1.0%) to vesicles formed in neutral saccharide solutions. Disaccharides (Table 3, I-K) and maltotriose (Table 3, L) show no significant difference in encapsulation efficiency and behave analogously to the monosaccharides.

The total surfactant concentration for each formulation was similar and fell in a range between 6.2 and 8.3 mM. The SDBS:CTAT mole ratio was also similar for all preparations, except glucose vesicles prepared in deionized water which gave a lower 55:45 ratio (Table 3, C). This is in accordance with the ratio observed when empty 60:40 w/w vesicles were formed and purified in deionized water (Table 2, F).

TABLE 3
Summary of vesicle-encapsulated sugar preparations.

| | | [Sugar], mM | | | | Vesicle |
|---|---|---|---|---|---|---|
| | | (in purified | EE | [Surfactant | mol % Ratio | Radius, |
| Entry | Sugar | vesicles) | (%)^{a} | Total], mM | SDBS:CTAT | (nm, PDI)^{b} |
| a | D-ribose | 0.28 ± 0.02 | 0.084 | 6.3 ± 0.1 | 63:37 | 72, 0.20 |
| b | D-glucose | 3.7 ± 0.8 | 1.1 | 7.5 ± 0.4 | 62:38 | 75, 0.17 |
| c | D-glucose (H₂O)^{c} | 2 ± 1 | 0.67 | 7.1 ± 0.7 | 55:45 | 178, 0.40 |
| d | D-galactose | 2.7 ± 0.7 | 0.82 | 7.6 ± 0.2 | 61:39 | 83, 0.20 |
| e | D-mannose | 2.9 ± 0.2 | 0.87 | 8.4 ± 0.2 | 64:36 | 74, 0.18 |
| f | α-D-methylglucoside | 0.6 ± 0.2 | 0.17 | 6.8 ± 0.2 | 63:37 | 75, 0.19 |
| g | D-glucosamine^{d} | 2.3 ± 0.2 | 3.5 | 12 ± 2 | 55:45 | 78, 0.23 |
| h | D-glucosamine-2-sulfate | 3.3 ± 0.7 | 1.0 | 7.0 ± 0.2 | 64:36 | 69, 0.06 |
| i | D-maltose | 4.1 ± 0.3 | 1.2 | 7.7 ± 0.3 | 62:38 | 80, 0.13 |
| j | D-cellobiose^{e} | 2.0 ± 0.4 | 1.2 | 7.8 ± 0.8 | 63:37 | 90, 0.15 |
| k | D-sucrose | 3.2 ± 0.4 | 0.86 | 6.5 ± 0.1 | 63:37 | 82, 0.12 |
| l | maltotriose | 2.2 ± 0.2 | 0.65 | 7.9 ± 0.2 | 63:37 | 95, 0.13 |

All were prepared in the presence of 500 mM sugar unless otherwise noted.
${}^{a} EE = \frac{{[ Sugar ]}_{purified} \times 1.5}{{[ Sugar ]}_{crude}} \times 100 ; the coefficient of 1.5 is due to dilution during purification .$ ^{b}Radii measured after 24 h.
^{c}Glucose vesicles prepared in deionized water instead of PBS 1X.
^{d}100 mM glucosamine was used instead of 500 mM due to vesicle instability at higher concentrations.
^{e}250 mM cellobiose was used instead of 500 mM during vesicle preparation due to insolubility.

The average radius of each SV preparation was also similar, falling into a range of 72-95 nm, except for glucose SVs prepared in deionized water which had a radius of 178 nm (Table 3, C), measured after 24 h. Furthermore, glucose vesicles prepared in deionized water were unstable with an increasing radius and PDI when monitored by DLS over time, whereas the other vesicle preparations displayed minimal variation in vesicle size over time. This result was at first surprising as catanionic SVs are expected to display increased stability in lower ionic strength solution due to a larger Debye length. It was proposed that the low surfactant ratio for vesicles formulated in deionized water (55:45; Table 1, D) is responsible for the observed anomaly. Only a 10% excess of SDBS exists in the vesicle bilayer when formed in water instead of the 20-25% excess observed in PBS 1× vesicle preparations. Accordingly, because catanionic SVs that have a surfactant molar ratio near unity are known to be less stable, the osmotic pressure differential incurred upon vesicle purification leads to instability.

It was previously reported that increasing glucose concentration in a decrease in EE; the highest efficiencies of incorporation were observed for the lowest initial sugar concentration. To see if the present system was analogous, catanionic SVs prepared at three glucose concentrations: 0.1, 0.2 and 0.5 M were analyzed. These results are nearly identical to what was previously known, showing that, while the amount of encapsulated glucose is less at lower starting glucose concentrations, a higher EE was observed with a 1.7-fold difference between vesicles prepared in 0.1 M glucose (EE=1.9%) and 0.5 M glucose (EE=1.1%). The origin of the diminishing return on EE at higher glucose concentrations may be due to higher osmotic pressure upon purification which would lead to an eventual plateau in EE above 0.5 M glucose as previously demonstrated

Release of Carbohydrates from SVs.

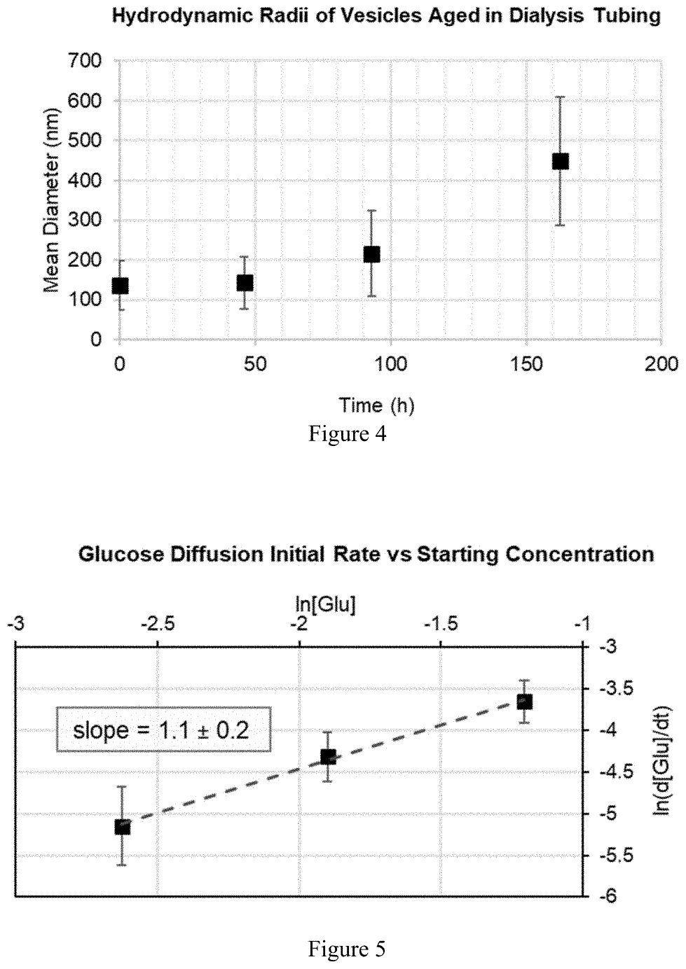

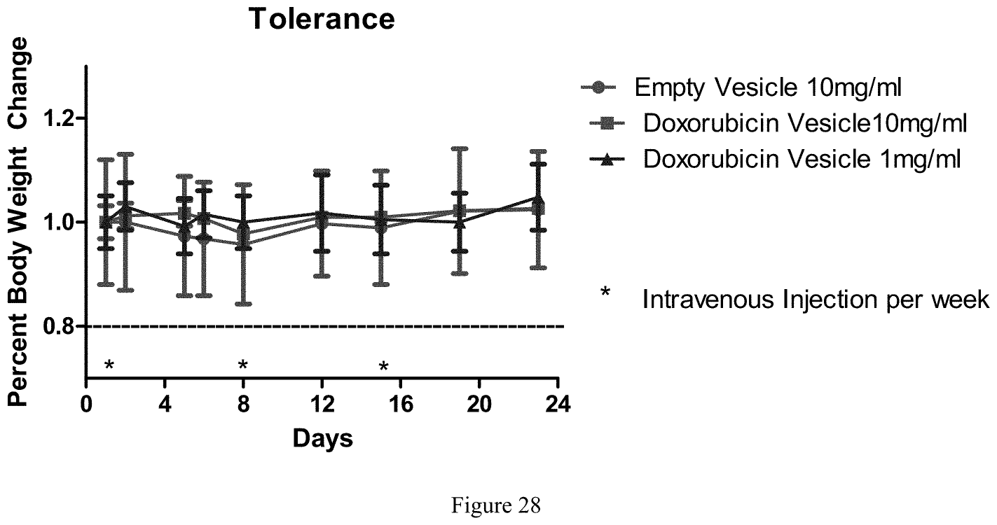

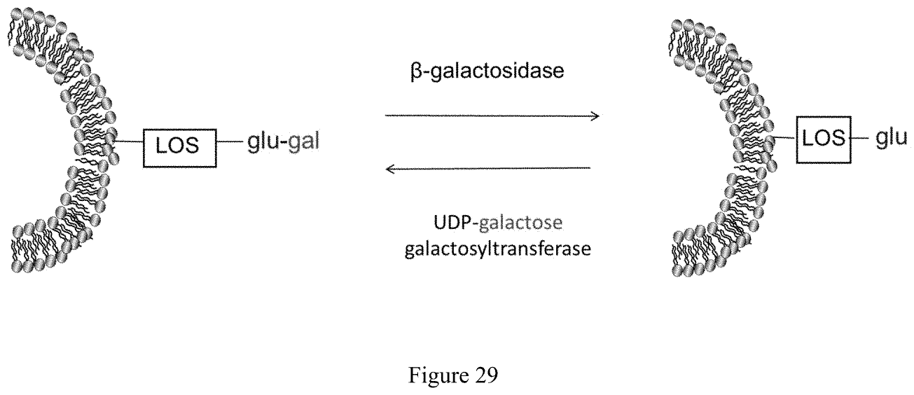

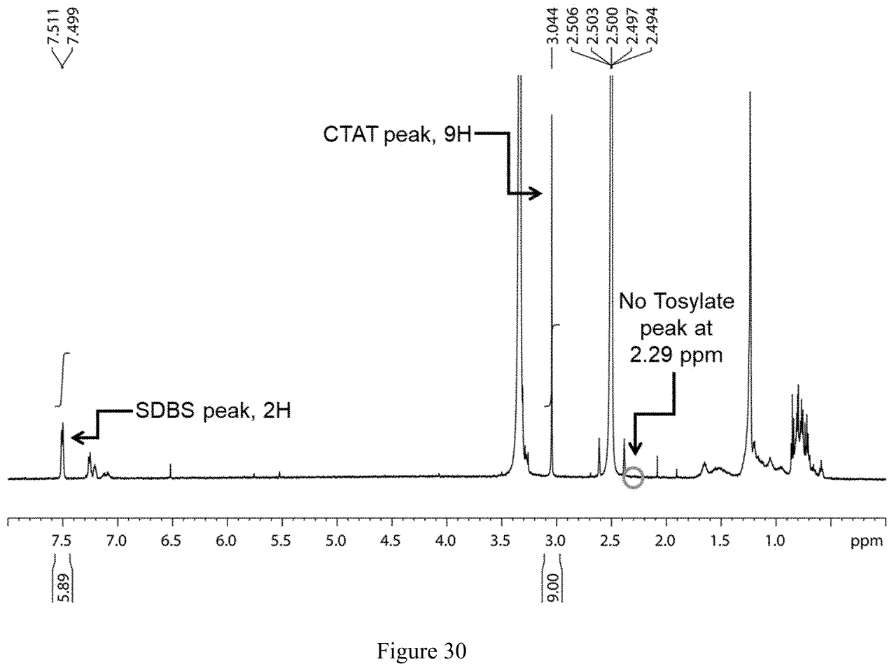

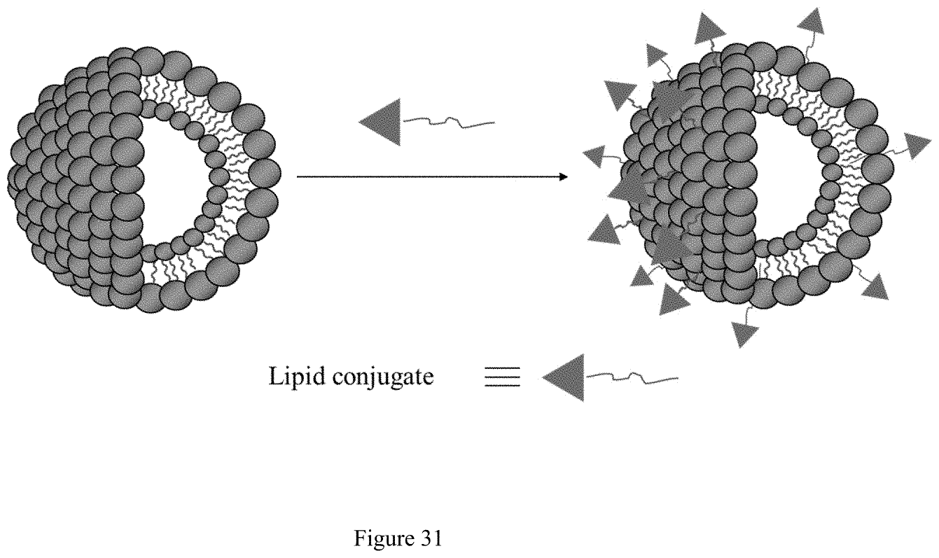

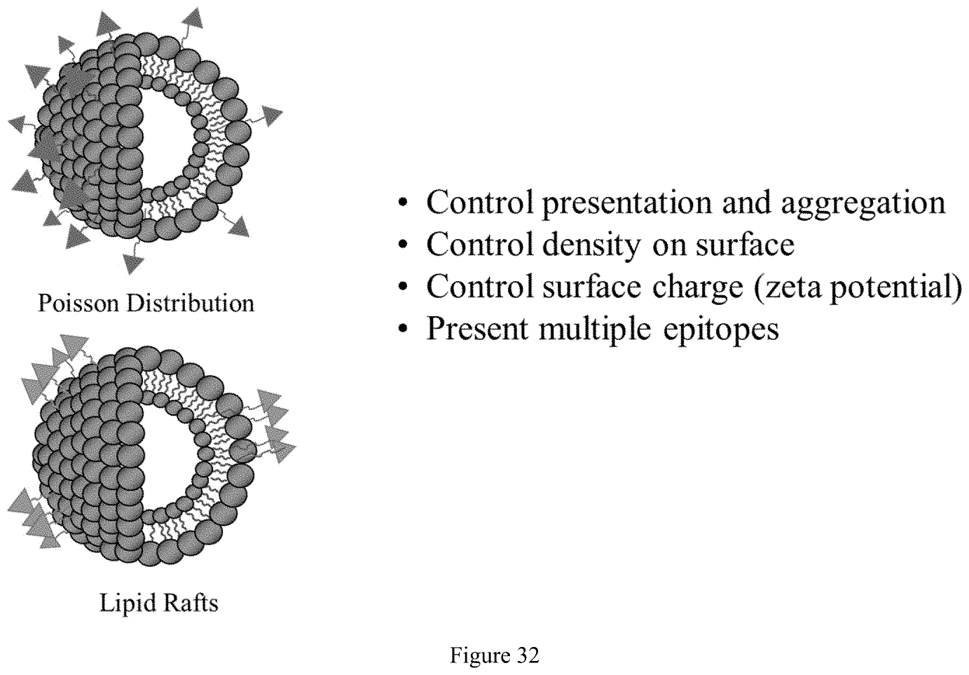

To assess the rate of encapsulated substrate release, dialysis was initially considered. However, when a solution of empty catanionic SVs was aged in dialysis tubing the mean diameter of the SVs increased dramatically after 50 h and a white precipitate was seen after 150 h (FIG. 4). Because the integrity of the SVs was compromised by the dialysis membrane during protracted exposure, an alternative analytical assay for carbohydrate release was developed. The assay was to remove aliquots from SVs-encapsulated formulations at time intervals and re-purify the aliquot by size-exclusion chromatography (SEC). Vesicle fractions elute in the initial fractions while release carbohydrate is retained on the column and elutes in the later fractions. The quantity of carbohydrate released was determined by ¹H-NMR (FIG. 28). Note that the purification of the aliquots by SEC required less than 5 min/sample and this delay did not significantly compromise the kinetic measurements in most instances.

Using the SEC method, the initial rate of glucose release was analyzed for vesicle encapsulated glucose solutions at varying concentrations. A first-order relationship was observed with the initial rate of glucose release from catanionic SVs prepared in 0.1, 0.2, and 0.5 M glucose solutions (FIG. 5). Therefore, a simple model with first-order release kinetics across the vesicle membrane (Eq. 1) was used, from which the rate law (Eq. 2) can be derived, where [Sugar]ᵢₙ and [Sugar]ₒᵤₜ are the sugar concentrations inside and outside the vesicle, respectively. Assuming the rate constants are identical in both directions across the vesicle membrane, and considering that the volume inside the vesicles only accounts for ˜1% of the total solution volume (i.e. [Sugar]ᵢₙ>>[Sugar]ₒᵤₜ for most of the experiment), then the diffusion of sugar into the vesicle can be ignored and Eq. 2 can be simplified to the pseudo-first-order Eq. 3.

$${[ Sugar ]}_{in} {[ Sugar ]}_{out} \qquad ( 1 )$$

$$\frac{- {d [ Sugar ]}_{in}}{dt} = {{k}_{1} [ Sugar ]}_{in} - {{k}_{- 1} [ Sugar ]}_{out} \qquad ( 2 )$$

$$\frac{- {d [ Sugar ]}_{in}}{dt} \approx {{k}_{1} [ Sugar ]}_{in} \qquad ( 3 )$$

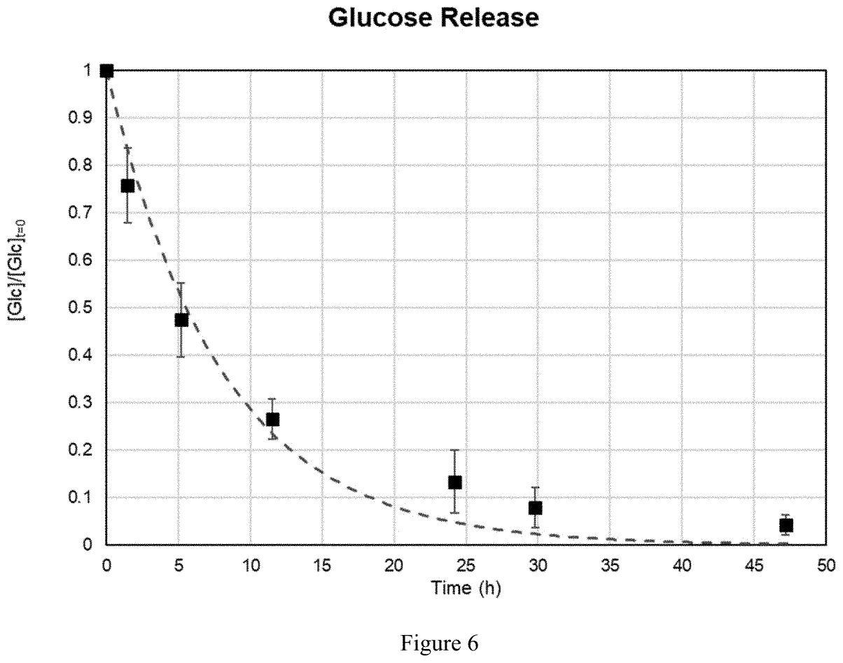

The release for each vesicle-encapsulated sugar preparation was monitored for at least two half-lives (monosaccharides) and at least one half-life (disaccharides and maltotriose). The release profile for each substrate was then fit to Eq. 3 by a least squares method, and the first order rate constant k₁ was calculated. The reported uncertainties represent the standard deviation in the measured k₁ of three separate batches of vesicle-encapsulated sugar. For monosaccharides (glucose, galactose, mannose, methyl D-glucopyranoside, glucosamine, and glucosamine-2-sulfate), the pseudo-first-order model (Eq. 3) was found to accurately describe substrate release. The release profile averaged from three batches of glucose vesicles is shown in FIG. 6, displaying a typical variation of about ±25% between batches.

The t_{1/2} was calculated from each k₁, and the permeability coefficient (P) was calculated using k₁ and the vesicle size (from DLS experiments) according to:

$${k}_{1} = \frac{PA}{{v}_{i}} \qquad ( 4 )$$

where vᵢ is the internal volume, and A is the surface area of the vesicle.

In general, the measured monosaccharide diffusion rates across the membrane of SDBS-rich catanionic SVs in PBS 1× are within the same order of magnitude as the rates observed for phospholipid and fatty acid liposomal formulations. For instance, the permeability coefficient for glucose across lecithin membranes is reported as 0.3×10⁻¹⁰ cm/s, while herein it was measured to be 0.82×10⁻¹⁰ cm/s for SDBS-rich, anionic SVs. Similar kinetics for glucose release were observed from vesicles prepared in both PBS 1× and deionized water (Table 4, B and C), though the instability of vesicles in deionized water (vide supra) led to a large uncertainty and prevented the accurate calculation of a permeability coefficient.

The fastest release for any sugar was observed for ribose (Table 4, A). The concentration of ribose fell below the limit of detection for the first aliquot taken, so the release rate could not be precisely determined. However, the half-life was estimated to be ≤0.1 h based on the amount of ribose which was released during the initial purification of ribose-SVs (Table 3, A). A rapid ribose diffusion rate is consistent with the large proportion (˜20%) of smaller furanose forms in aqueous solutions of ribose.

The neutral hexoses (Table 4, B-E) displayed half-lives ranging from 3 to 6 h, with glucose being slowest and mannose fastest. The same trend has been observed for fatty acid membranes. Despite being a similar size, the lipophilic α-D-methylglucoside diffuses through the vesicle bilayer an order of magnitude faster than the other monosaccharides (Table 4, F), in accordance with Overton's rule. This result also supports the idea that the ability to access a straight chain form in solution does not lead to an increased rate of SV membrane diffusion for monosaccharides.

TABLE 4
Rates and permeability coefficients for the release of
monosaccharides.

| | | First Order | | Permeability |
|---|---|---|---|---|
| | | Rate Constant | Half-life | Coefficient P |
| | | k (h⁻¹) | t_{1/2} (h) | (×10⁻¹⁰ cm/s) |
| a | D-ribose | — | ≤0.1 | ≥13 |
| b | D-glucose | 0.12 ± 0.03 | 6 ± 1 | 0.82 |
| c | D-glucose (H₂O)^{a} | 0.12 ± 0.05 | 5 ± 3 | — |
| d | D-galactose | 0.16 ± 0.02 | 4.3 ± 0.7 | 1.2 |
| e | D-mannose | 0.26 ± 0.06 | 2.5 ± 0.7 | 1.9 |
| f | α-D-methylglucoside | 3.7 ± 0.5 | 0.20 ± 0.03 | 25 |
| g | D-glucosamine^{b} | 0.11 ± 0.01 | 5.3 ± 0.4 | 0.96 |
| h | D-glucosamine-2- | 0.29 ± 0.06 | 2.4 ± 0.5 | 1.9 |
| | sulfate | | | |

Experiments were performed in triplicate at 25° C. in PBS 1× solution with a pH of 7.4 using SDBS rich vesicles prepared with a 60:40 w/w surfactant ratio with a total surfactant concentration of 1% w/w and total sugar concentration of 500 mM.
^{a}Performed in water instead of PBS 1×.
^{b}D-glucosamine was performed at 100 mM sugar concentration due to vesicle destabilization at higher concentrations.

Surprisingly, glucosamine, a cation at pH 7.4, as well as anionic glucosamine-2-sulfate showed little difference in release rate (Table 4, G and H) from the neutral hexoses with only a 2-fold difference in rate compared to each other. In both cases, diffusion across the leaflet is likely aided by the high ionic strength of PBS 1× that limits the electrostatic effect of charged species (Debye length is inversely proportional to ionic strength), lowering the energy barrier for the close approach of glucosamine-2-sulfate to the vesicle bilayer as well as the dissociation of glucosamine from the bilayer into the bulk medium.

The kinetics for release of disaccharides and maltotriose, a trisaccharide, are significantly slower than the monosaccharides, with initial rates that are roughly an order of magnitude reduced, despite being only twice the molecular weight. This retardation of the kinetics of release suggests that catanionic SVs may be a desirable vehicle for sequestering and delivering hydrophilic carbohydrate derivatives (e.g., heparin) when compared with their more common liposomal counterparts.

Unlike the monosaccharides, the profiles for release for disaccharides (maltose, cellobiose, and sucrose) and maltotriose does not follow first-order kinetics. To test if an equilibrium effect may favor diffusion into the vesicles and be responsible for the deviation from first-order kinetics (i.e. if k₋₁>>k₁ in Eq. 2), a solution of empty SVs was incubated in 4 mM D-maltose solution for three weeks. Upon purification, it was observed that the vesicles had only trapped 0.2 mM of the maltose, which represents 5% EE. This incorporation is higher than the 1.2% EE when prepared in 500 mM D-maltose solution (Table 3, I) was used for the formulation of maltose-SVs by the standard methodology, and suggests that an inverse relationship between EE and sugar concentration (FIG. 3) is operative. For di- and trisaccharides a second-order profile was measured as described by Eq. 6.

$$\frac{- {d [ Sugar ]}_{i n}}{d t} \approx {{k}_{1}^{′} [ Sugar ]}_{i n}^{2} \qquad ( 6 )$$

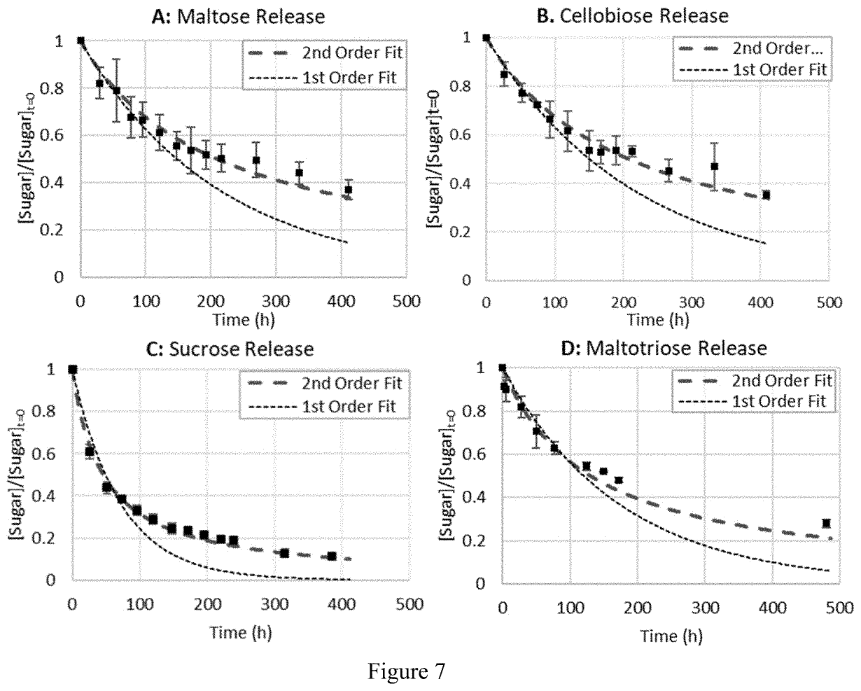

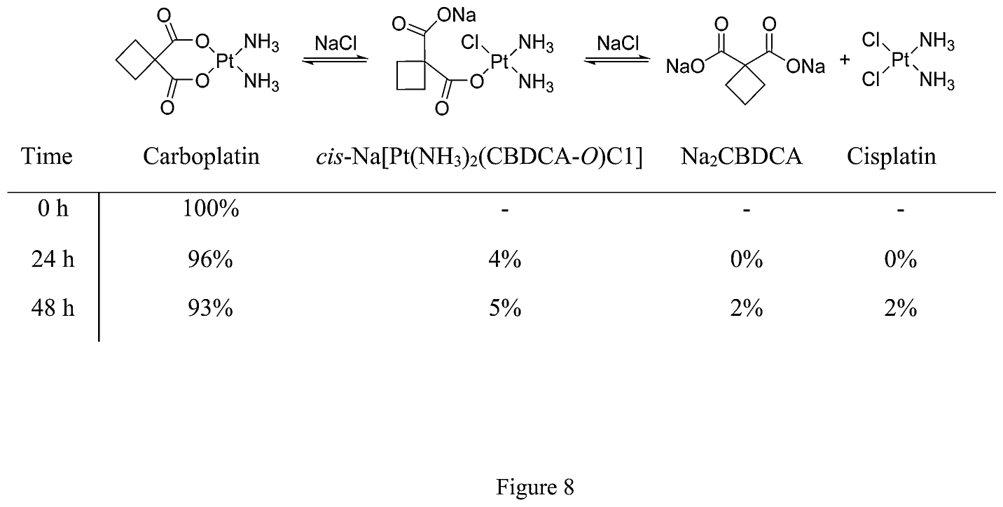

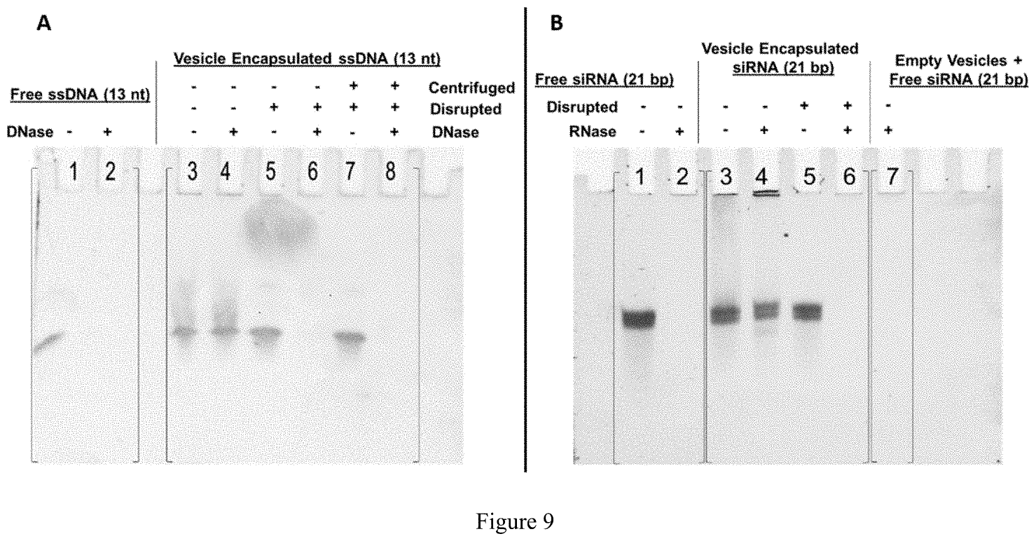

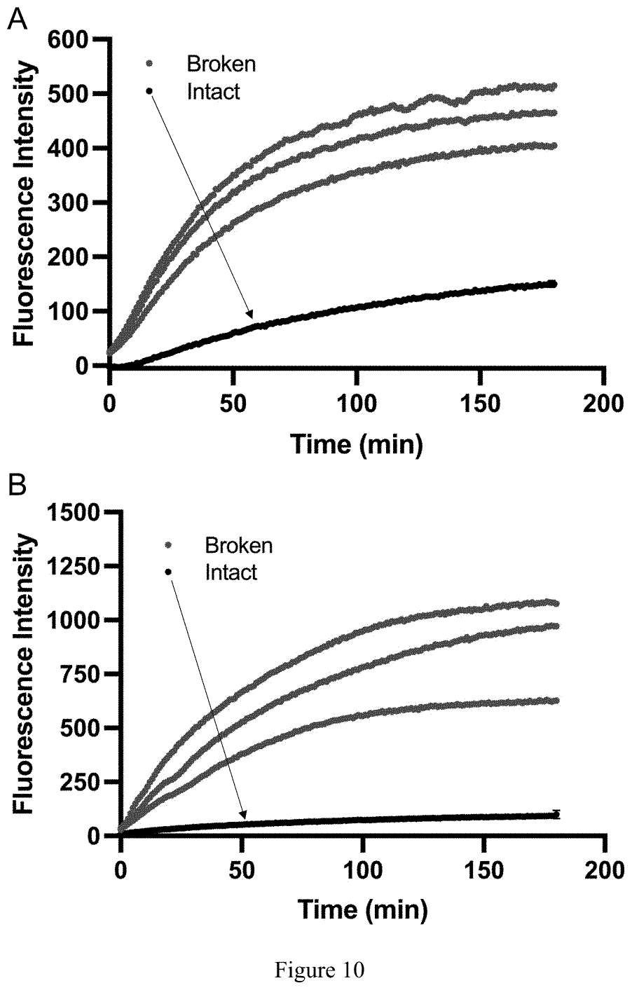

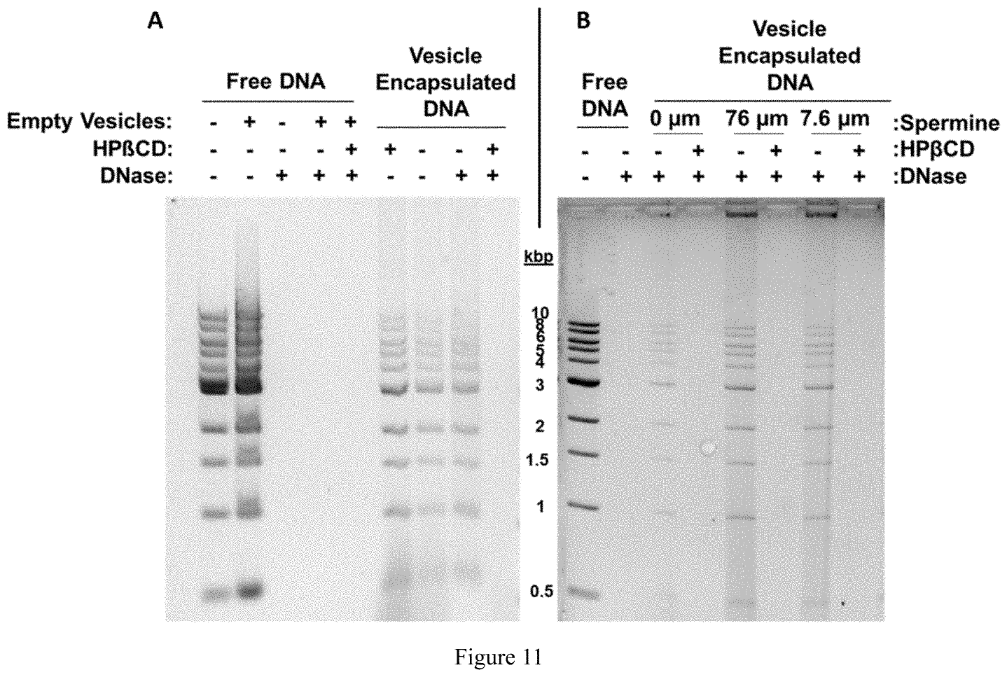

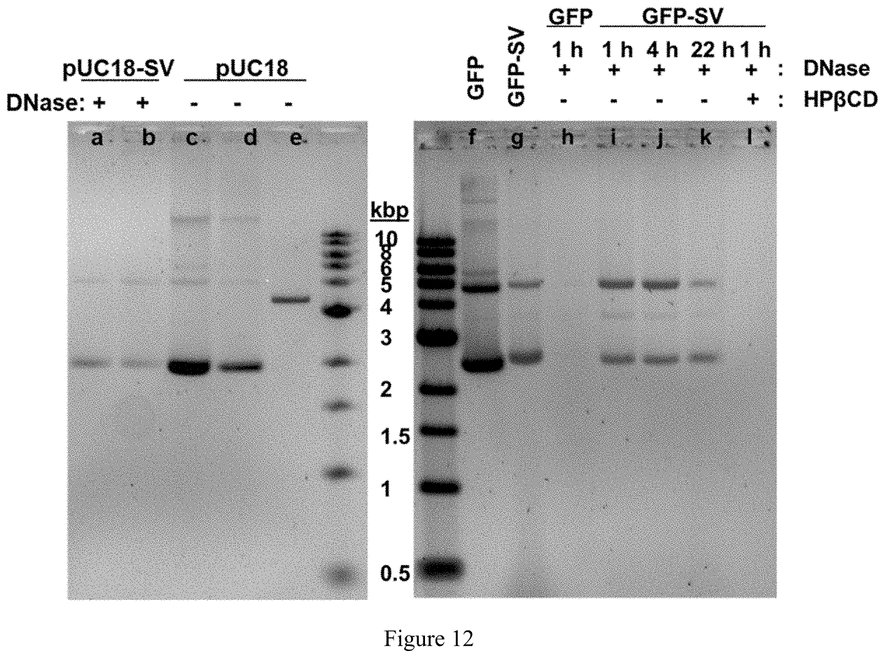

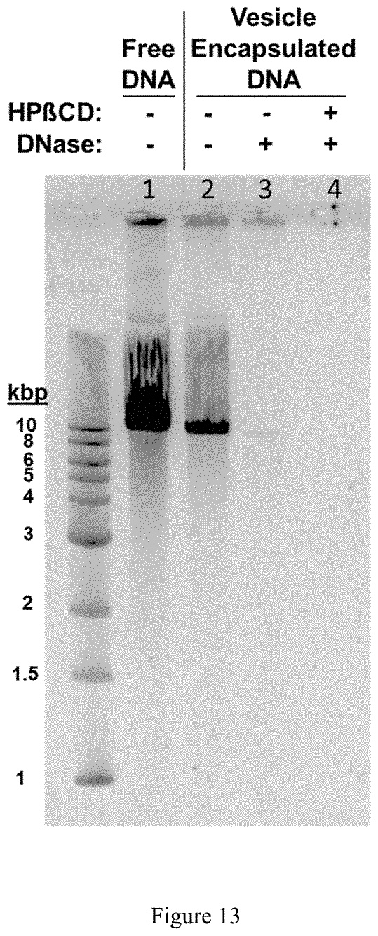

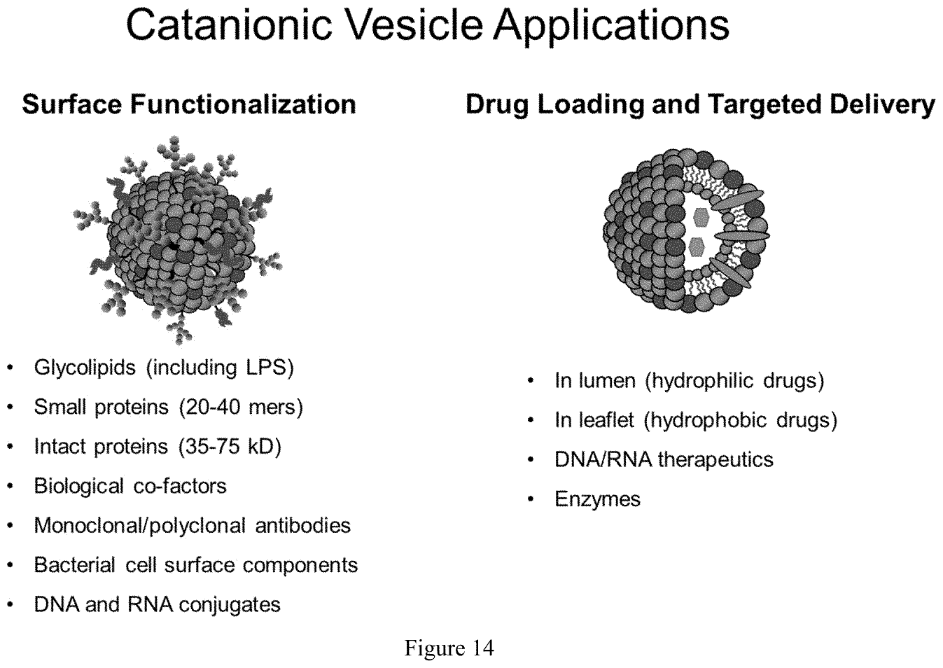

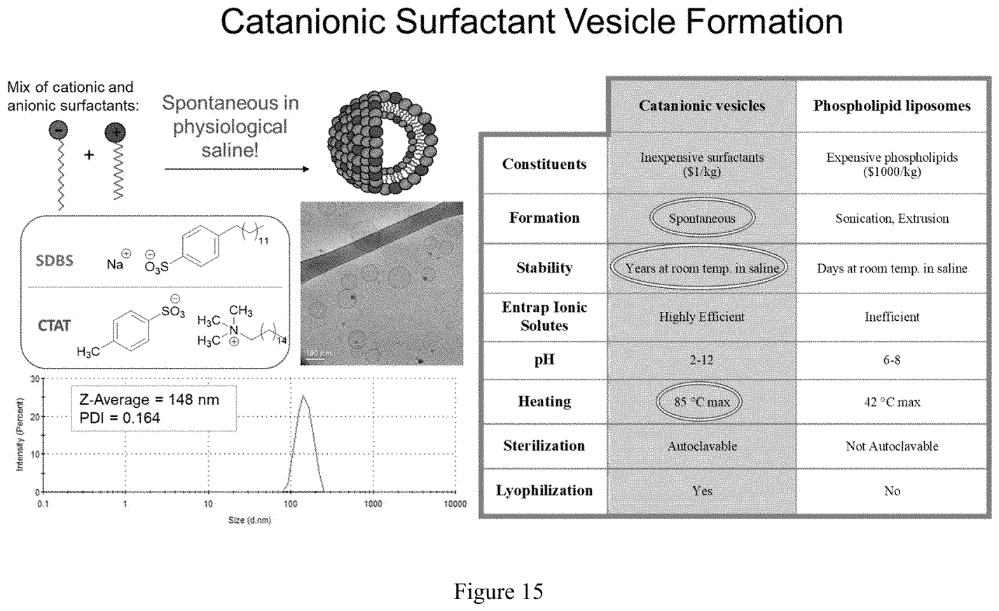

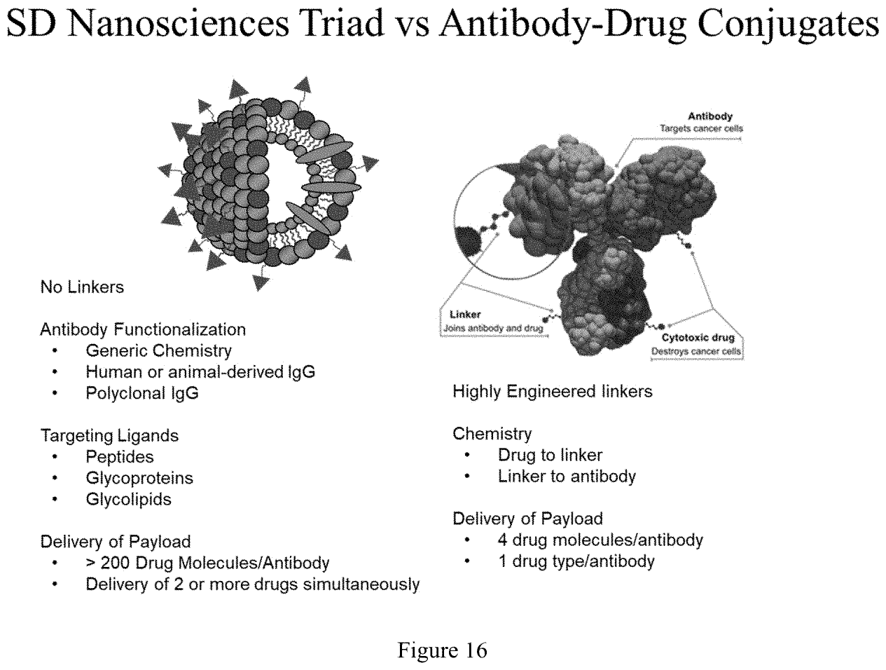

It is proposed that the observed second-order kinetics for the release of disaccharides and maltotriose is an indication that these substrates are released by a different diffusion mechanism. For example, sucrose is known to displace the ordered water shell at the vesicle headgroup-solvent interface and may therefore aid the approach of additional sucrose molecules to the inner vesicle surface during diffusion. In addition, maltose and sucrose are known to form aggregates in solution and these aggregates may diffuse across the membrane as a dimeric or oligomeric species. In all four cases (maltose, cellobiose, sucrose, and maltotriose) the second-order rate law (Eq. 6) is observed for release (FIG. 7, A-D).

The second order rate constants derived from the fittings in FIG. 7 are summarized in Table 5 along with permeability coefficients derived from initial rates. Maltose was the slowest to diffuse across the SV membrane with a second order rate constant of 1.2±0.3 L/mol·h. Cellobiose, with a β(1→4) glycosidic bond (vs. an α(1→4) glycosidic bond in maltose) has a more linear conformation in solution and diffuses about twice as quickly as maltose. Sucrose leaked 5.6-fold faster than maltose (Table 5, C), likely in part due to its smaller furanose portion and possibly its inability access a linear chain form for either subunit.

Maltotriose has been reported to diffuse at half the rate of maltose through phosphatidyl choline membranes. However, quite surprisingly, maltotriose leaked across the SV leaflet faster than any of the disaccharides analyzed, despite being ˜50% larger (Table 5, D). The maltose-SVs formed in 500 mM maltotriose solution (employing standard SVs formulation methodology) had the largest radius measured by DLS of any other PBS 1× preparation analyzed (95 nm; Table 3, L), hinting at possible destabilization of the SV system. This destabilization of the membrane may result in faster diffusion kinetics because pores or holes may be formed. This was unexpected.

TABLE 5
Second order rate constants and permeability coefficients
for the release of disaccharides and maltotriose.

| | | Second Order | Permeability |
|---|---|---|---|
| | | Rate Constant | Coefficient |
| Entry | Sugar | k’ (L/mol · h) | P (×10⁻¹⁰ cm/s)^{a} |
| a | D-maltose | 1.2 ± 0.3 | 0.031 |
| b | D-cellobiose | 2.6 ± 0.5 | 0.043 |
| c | D-sucrose | 6.7 ± 0.4 | 0.12 |
| d | D-maltotriose | 7.6 ± 0.8 | 0.18 |

Sugar concentrations measured by ¹H-NMR as described in the supplemental material.
^{a}Permeability coefficients derived from initial rates and DLS data

Encapsulation and Release of Chemotherapeutics.

The ease of synthesis and exceptional stability of SDBS-rich, anionic SVs make them an attractive alternative to classical liposome formulations for drug delivery system. When vesicles are combined with surface modifications such as glycans, monoclonal antibodies, or folate conjugates then targeted drug delivery may be realized. Targeted drug delivery can lower therapeutic dose and slow systemic drug accumulation, and is therefore especially attractive as a strategy for mitigating the toxicity of anti-cancer drugs without impacting efficacy. Based on the success of encapsulation of carbohydrate derivatives, the encapsulation and release kinetics of four common anti-cancer drugs, doxorubicin, pemetrexed, carboplatin, and cisplatin, was investigated.

Table 6 summarizes the EE and drug concentration achieved with each of the chemotherapeutic agents under the standard SDBS-rich SVs formulation method. The 60% EE measured for doxorubicin agrees well with the value reported previously for doxorubicin/SDBS/CTAT vesicles in deionized water, and is markedly higher than for pemetrexed (0.80%, Table 6 B), carboplatin (1.9%, Table 6 C), or cisplatin (2.1%, Table 6 D). Since the aqueous vesicle lumen represents only about ˜1% of the total solution volume, the excellent doxorubicin encapsulation efficiency is consistent with a thermodynamic preference associated with the vesicle. Two different effects likely give rise this preference: (1) In PBS 1× buffer (pH=7.4), the majority of doxorubicin (pKa=8.4) is in the cationic form and can bind electrostatically to the negatively charged surface of the vesicle bilayer and (2) the freebase neutral form has a predicted Log P of 1.3, so favoring solvation in the hydrophobic microenvironment inside the bilayer. The significantly lower encapsulation efficiencies measured for pemetrexed and the two platinum drugs (Table 6, B-D) are similar to the values measured for saccharide encapsulation and likely represent a good approximation of the volume percent of solution contained within the vesicle lumen.

TABLE 6
Encapsulation and release of chemotherapeutic drugs in catanionic SVs.

| | | | | First | | |
|---|---|---|---|---|---|---|
| | [Drug], mM | [Drug], mM | | Order Rate | | Vesicle |
| | (in crude | (in purified | EE | Constant | Half-life | Radius, |
| Drug | mixture) | vesicles) | (%)^{a} | k (h⁻¹) | t_{1/2} (h) | (nm, PDI) |
| Doxorubicin | 0.34 | 0.15 ± 0.02 | 61 | — | >300 | 91, 0.25 |
| Pemetrexed | 84 | 0.45 ± 0.09 | 0.80 | 0.015 ± 0.001 | 47 ± 4 | 73, 0.17 |
| Carboplatin | 40 | 0.5 ± 0.1 | 1.9 | 0.039 ± 0.004 | 18 ± 2 | 74, 0.19 |
| Cisplatin | 3.3 | 0.05 ± 0.01 | 2.1 | 0.78 ± 0.06 | 0.89 ± 0.07 | 76, 0.16 |

Drug concentrations in purified vesicle preparations were analyzed by ¹H-NMR (doxorubicin, pemetrexed, carboplatin) and ICP-AES (cisplatin) as described in the supplementary material.
Drug diffusion rates were analyzed by ¹H-NMR (doxorubicin, pemetrexed, and carboplatin) and ICP-AES (cisplatin) by the same manner as the mono- and disaccharides.
Half-lives were calculated from the first order rate constant measured in three different batches of vesicles except for cisplatin which only encompasses two batches.
$\begin{matrix}{}^{a} EE = \frac{{[ Drug ]}_{purified} \times 1.5}{{[ Drug ]}_{crude}} \times 100 with the coefficient of 1.5 compensating for vesicle dilution on the \\ SEC column .\end{matrix}$

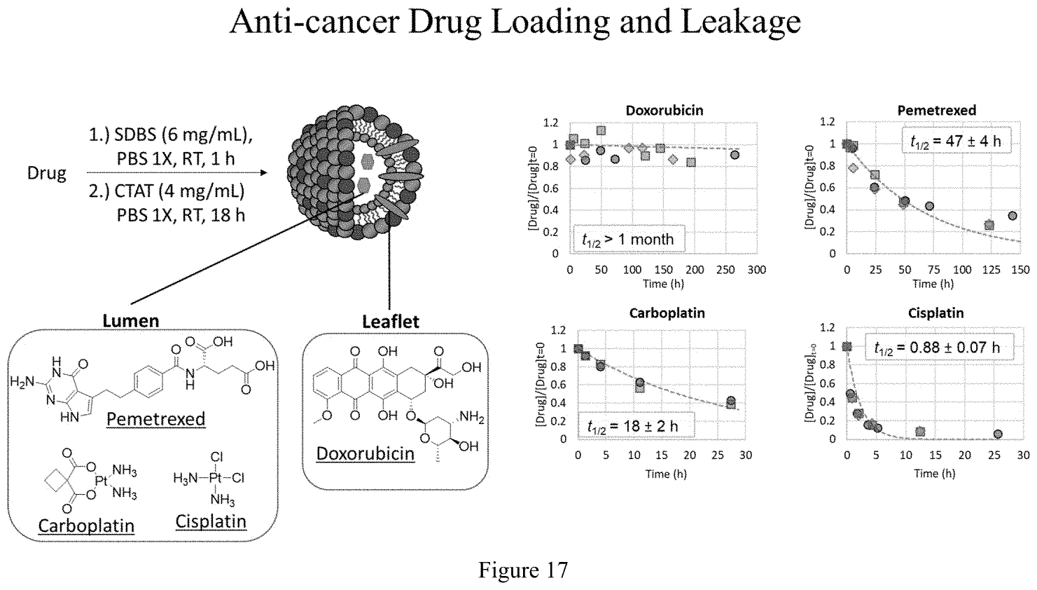

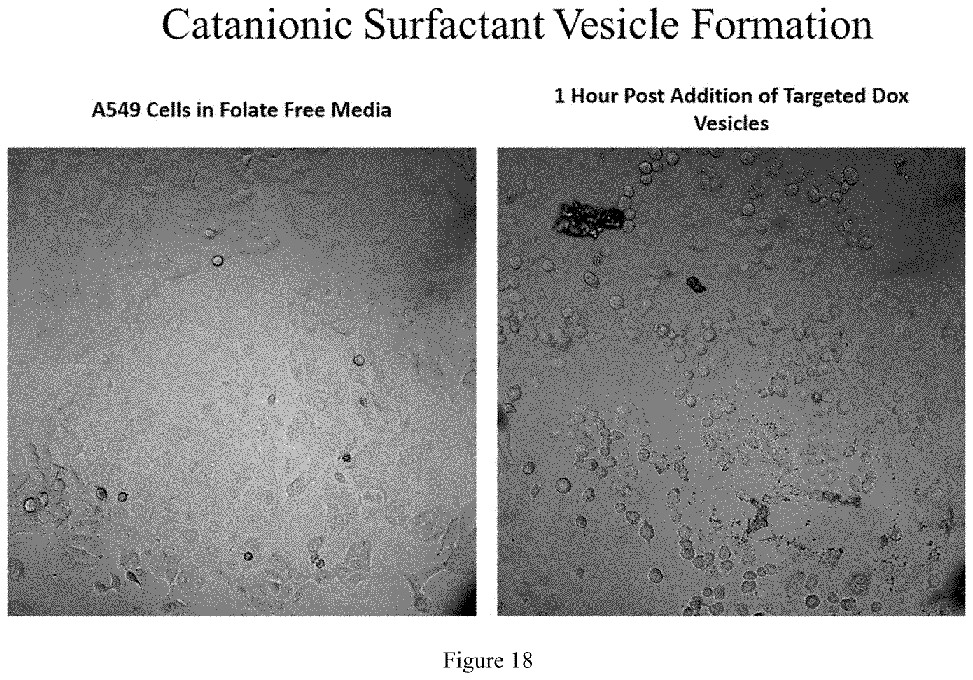

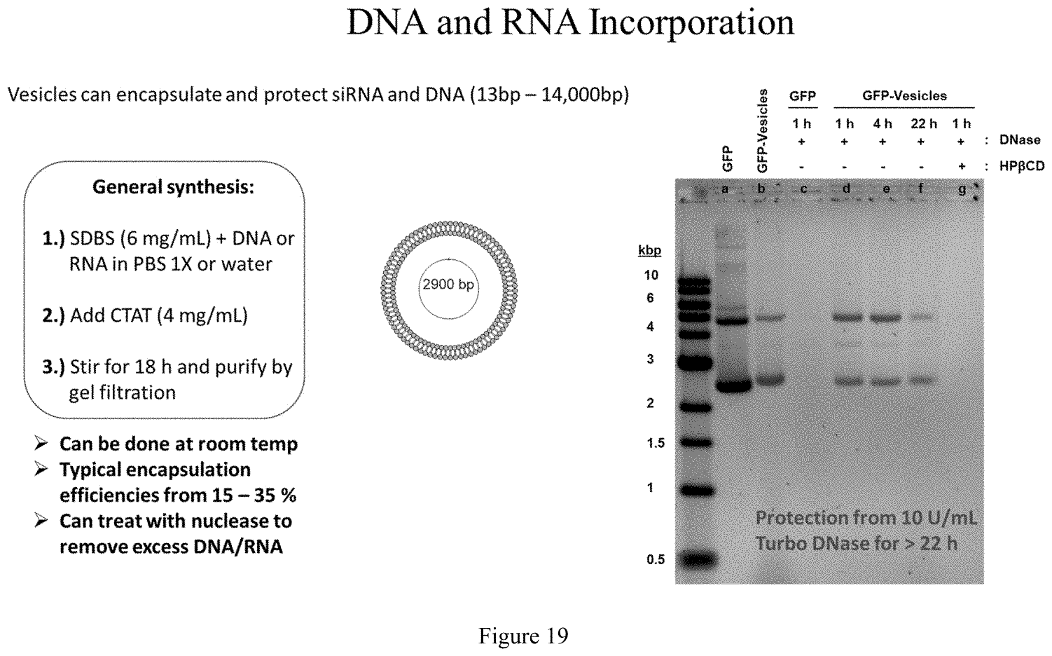

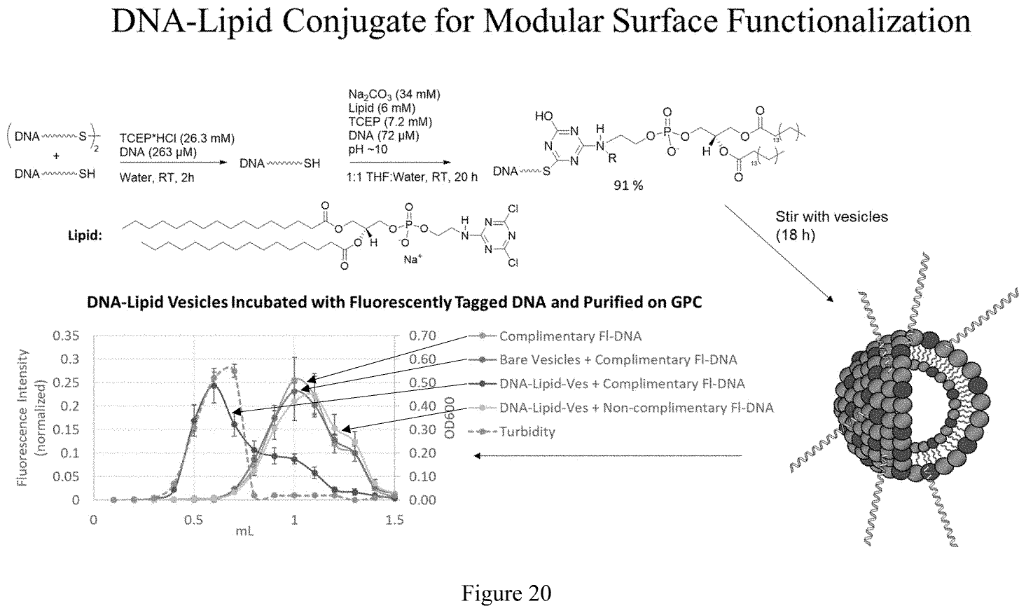

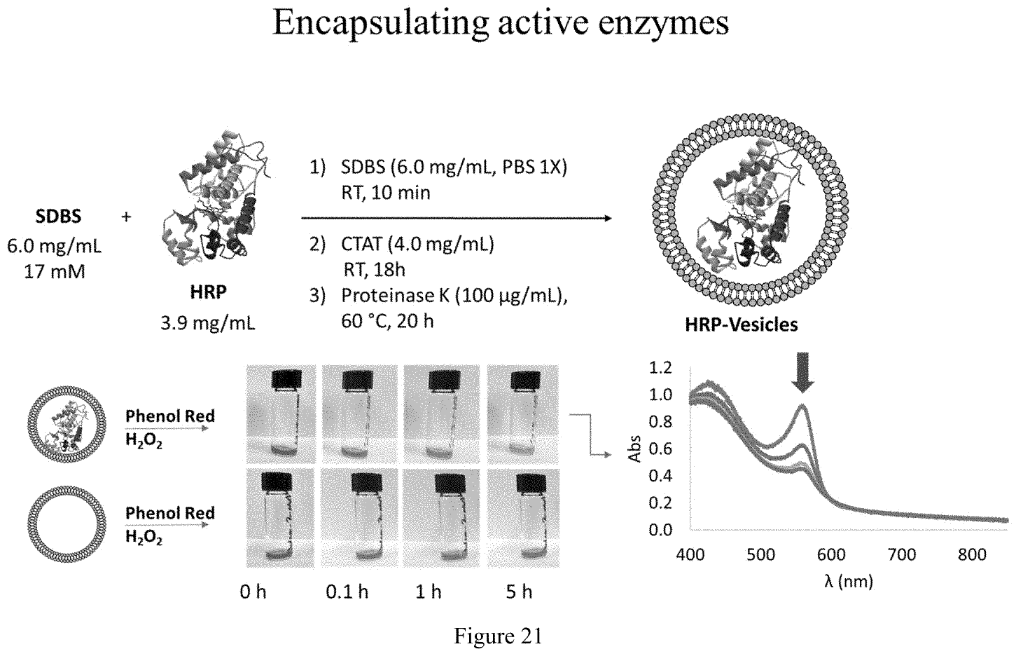

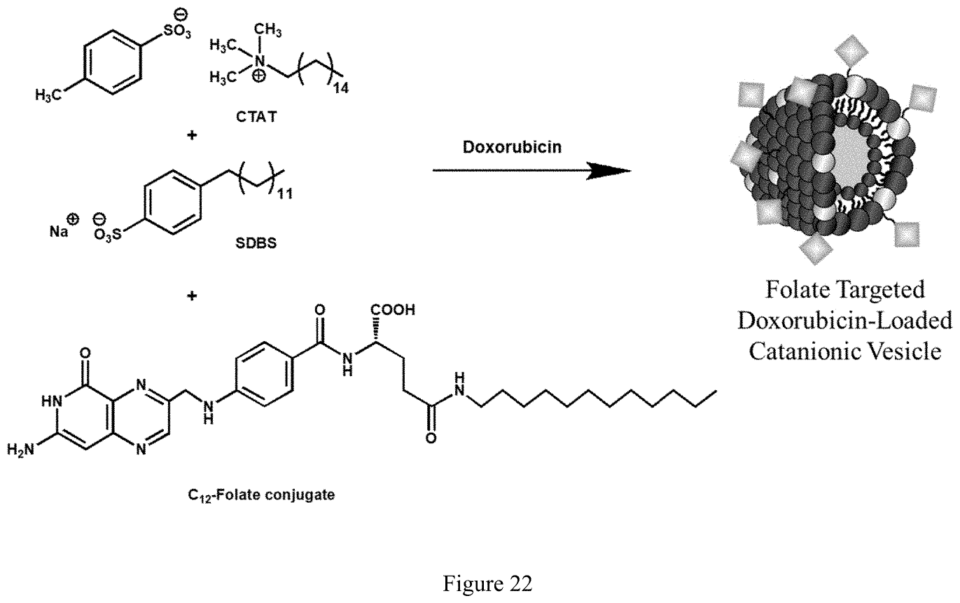

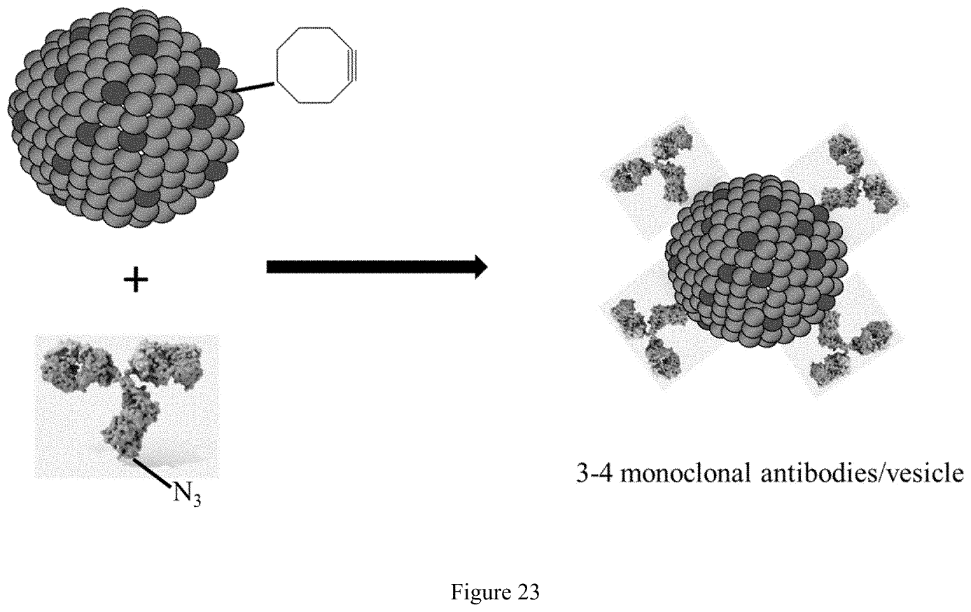

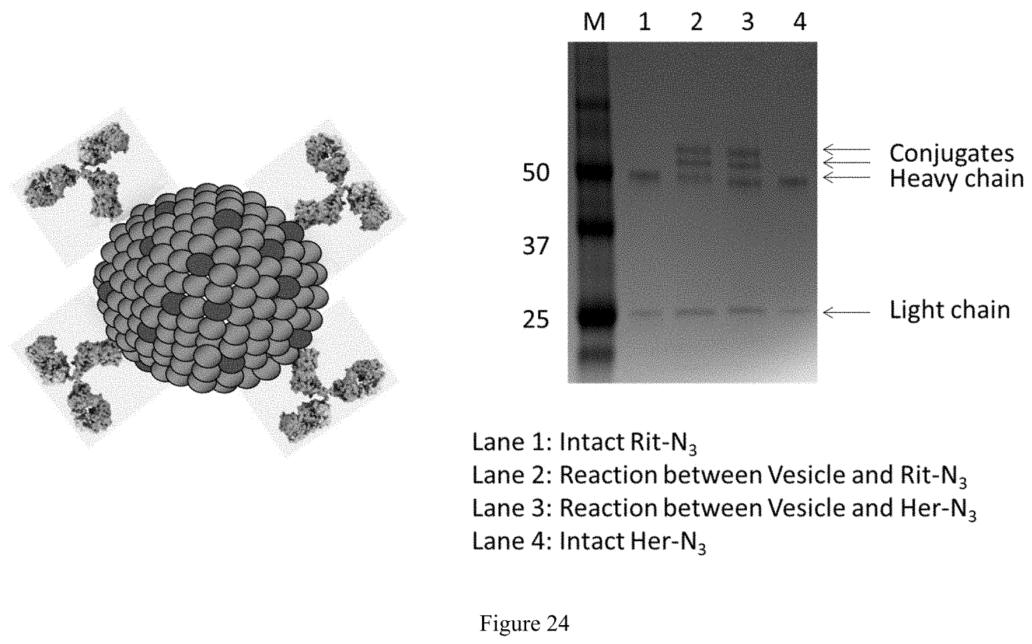

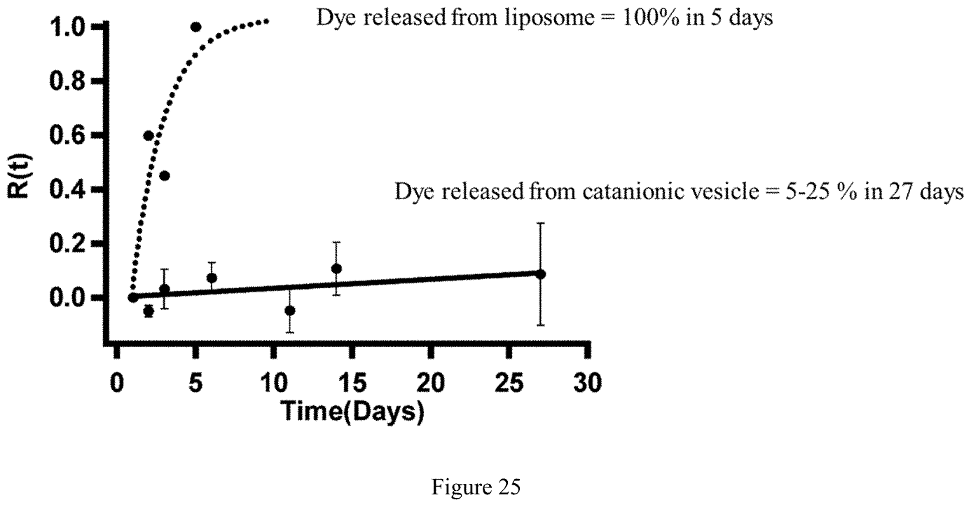

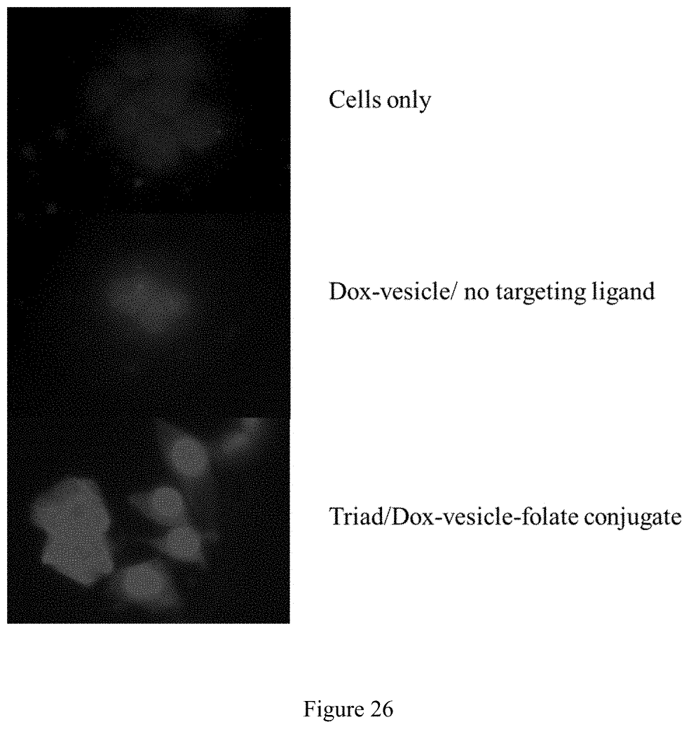

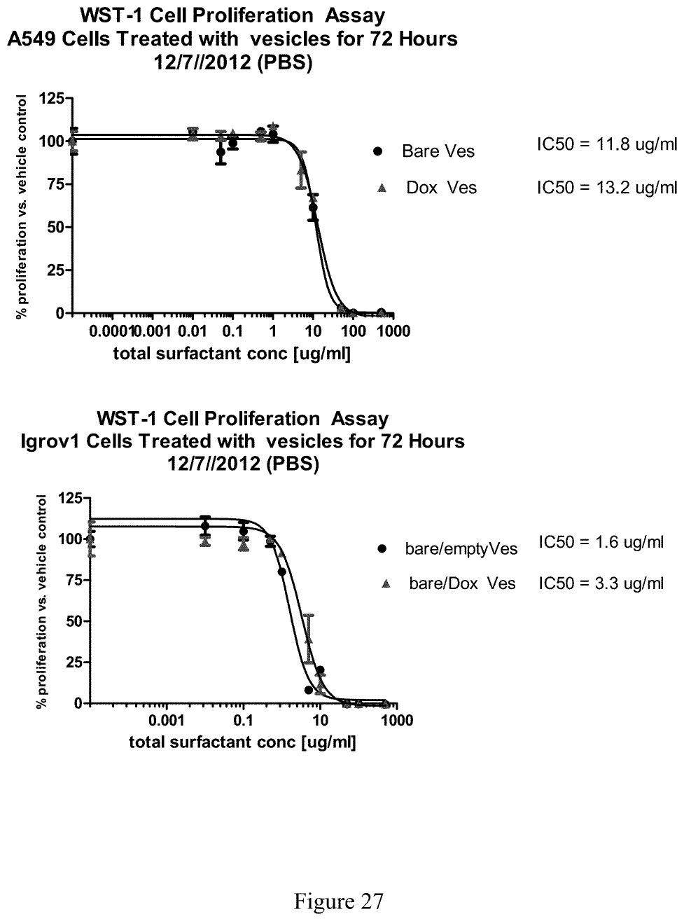

As can be seen in FIG. 17 (right), the four drugs provide a broad range of release profiles. Doxorubicin showed no release, within detection limits, after 11 days. The slow release of doxorubicin is readily explained by assuming that the highly hydrophobic doxorubicin is embedded into the hydrophobic region of the leaflet and is not readily released into the aqueous environment.

The three remaining chemotherapeutics are highly hydrophobic by comparison, and pass more readily across the leaflet. Pemetrexed was released with t_{1/2}=47±4 h. The slow release of pemetrexed compared to the platinum drugs or monosaccharides analyzed in this work is likely due to both its higher molecular weight as well as its negative −2 charge at pH 7.4 which would result in an energetic barrier toward approaching the negatively charged bilayer surface, though the high ionic strength of PBS buffer might serve to mitigate this effect to an extent.

Carboplatin is similar in molecular weight to monosaccharides and is moderately water soluble, so it was anticipated that the release kinetics for carboplatin would be analogous with the monosacharides. However, carboplatin is released 3-fold more slowly than glucose (t_{1/2}=18±2 h vs. 6±1 h) and 20-fold more slowly than the similar platinum drug cisplatin (t_{1/2}=0.88±0.07 h). Probably, the hydrophobic sidechain of carboplatin plays a key role in the release kinetics by strongly associating with the leafet of the SVs. Carboplatin is known to reversibly form association complexes at high concentrations, such as those found within the vesicle, and these larger oligomeric structures retard the release of carboplatin vs. cisplatin.

The question arises as to whether carboplatin is released from the vesicle in its native form, especially since the conversion of carboplatin to cisplatin in saline is well known and cisplatin leaks much more quickly (FIG. 17, right). Analysis of a 40 mM solution of carboplatin (same as inside the vesicles) in PBS 1× revealed that 93% remained unreacted after 48 h at room temperature, and only 2% had been converted to cisplatin. This result is consistent with the slow rates of carboplatin ligand exchange in saline at 37° C. (k_{obs}=7.7×10⁻⁷ s⁻¹, t_{1/2}≈10 days). Considering well over 50% of the carboplatin had leaked out of the vesicles at the 48 h mark, it was concluded that the majority of carboplatin diffusing out of the catanionic SVs must be in its unreacted form, not converted to cisplatin.

Finally, cisplatin is released more rapidly than the other chemotherapeutics expressly measured herein. Presumably, several factors are in play: the molecule has a low molecular weight, does not carry a charge, and is hydrophobic (as indicated by its limited solubility in water (3.3 mM concentration in buffer compared to 40 mM for carboplatin) hindering its ability to self-associate and form larger complexes. Hydratization of cisplatin to give the ionic cis-[Pt(NH3)₂(H₂O)Cl]⁺ is suppressed in PBS 1× (NaCl=154 mM) and so it is expected that cisplatin is released in its native form.

### CONCLUSIONS

In summary, anion-rich catanionic SVs prepared from SDBS and CTAT are stable in a variety of cell growth media and buffers including PBS 1× solutions containing high concentrations of carbohydrate. Upon purification SVs sequester saccharide with EEs of ˜1%, representative of the volume percent of solution contained within the hydrophilic SVs lumen. Monosaccharides are released from the SVs in a first-order process dependent on their respective molecular weights and lipophilicity. The kinetics of release decreases by an order of magnitude for di- and trisaccharides. In addition, the kinetics of release shifts from a first-order to a second-order process. It is proposed that aggregation of the higher saccharides in the lumen plays a key role in the shift of the kinetic profile.

Anionic, catanionic SVs were also shown to sequester a variety of hydrophobic and hydrophilic chemotherapeutic agents. The EE was close to ˜1% for the hydrophilic drugs but much higher for doxorubicin (61%) suggesting this highly hydrophobic compounds partitions into the hydrophobic vesicle bilayer microenvironment. The kinetics of release for these compounds varied widely; they were determined to fall between t_{1/2}=0.89 to >300 h. This high degree of variability in the kinetics was again due to differences in molecular weight of the compound and lipophilicity as well as the ability to form self-association complexes in the case of carboplatin.

The present disclosure has further defined the kinetics of release for small neutral and charged molecules across the leaflet of catanionic SVs. It was also shown that catanionic SVs successfully sequester and slowly release common chemotherapeutic drugs on timescales amenable to drug delivery applications. Catanionic SVs are an alternative to conventional liposomes for controlled release of chemotherapeutics and future studies should include the in vitro or in vivo application of anion-rich catanionic SVs to deliver drug payloads to cells.

Abbreviations: PBS 1×—phosphate buffered saline, pH 7.4 (Corning, Cat #21-040-CMR); MeOH—methanol; EtOH—ethanol; SDBS—sodium dodecylbenzene sulfonate (Sigma, Cat #289957); CTAT—cetyl trimethyl ammonium tosylate (Sigma, Cat #C8147); FRET—Forster resonance energy transfer; Milli-Q—deionized water (18.2 megaohm ionic purity); PAGE—polyacrylamide gel electrophoresis; HPβCD—2-hydroxypropyl-β-cyclodextrin (Matrix Scientific, Cat #202446).

Synthesis of Catanionic Vesicle Formulations.

All glassware was oven dried at 200° C. to destroy any nuclease. Nuclease free Milli-Q water was used, and all buffers were purchased nuclease free. CTAT was recrystallized from methanol/acetone.

Synthesis of bare empty vesicles: To a 20 mL scintillation vial was added 60 mg SDBS and 40 mg CTAT followed by 10 mL PBS 1× along with a Teflon coated stir bar. The mixture was stirred at room temperature for 18 h. To purify, a 1.0 mL aliquot was removed and purified by gel filtration according to the 1.0 mL crude vesicle purification procedure outlined below, yielding 1.5 mL of purified vesicles.

Synthesis of 13 mer ssDNA Vesicles: A one-dram vial was charged with 6.0 mg SDBS and a small Teflon coated stir bar. To the vial was then added 500 μL 1.6 g/L ssDNA (5′-GGACAGCTGGGAG (SEQ ID NO:1)) followed by 500 μL PBS 1×. The mixture was stirred until all SDBS had dissolved (˜15 min). The solution of SDBS/DNA was then transferred to a glass vial containing 4.0 mg CTAT. The resulting mixture was stirred for 18 h at room temperature. The crude vesicles were then purified according to the 1 mL crude vesicle purification procedure outlined below, yielding 1.5 mL of purified vesicles.

Synthesis of 21 bp siRNA Vesicles: A one-dram vial was charged with 6.2 mg SDBS and a small Teflon coated stir bar. A second one-dram vial was charged with 4.0 mg CTAT. Both vials were autoclaved for 30 minutes followed by a 10 minute drying cycle. To the vial containing SDBS was added 250 μL from a stock solution of 21 bp siRNA (5′-GUG-UAU-CCA-ACA-CGG-AUC-CUC (SEQ ID NO:2)) in PBS 1× (188 μM, 2.52 g/L) followed by 750 μL PBS 1×. The mixture was then stirred at room temperature until all SDBS had dissolved and the solution became clear (˜15 min). The SDBS/siRNA solution along with the stir bar was then transferred to the vial containing CTAT and stirred at room temperature for 45 minutes until all the solid CTAT had dissolved and the solution became opaque. The vial was then placed in a 4° C. fridge and stirred for 18 h. The entire 1 mL volume was then purified according to the 1 mL crude vesicle purification procedure outlined below, yielding 1.5 mL of purified vesicles.

Synthesis of FRET pair DNA vesicles: DNA duplexes were prepared by combining 60 μM fluorescein labeled strand with 75 μM complementary quencher strand (see below for DNA synthesis). To anneal, the mixture was heated at 95° C. for 2 min and allowed to cool to room temperature. 100 μL was then mixed with 900 μL PBS 1× (10× dilution). The 1.0 mL diluted DNA solution was then added to a one-dram glass vial containing 6.0 mg SDBS and a small Teflon coated stir bar. The mixture was stirred until all SDBS had dissolved (˜20 min). The SDBS/DNA solution was then transferred, along with the stir bar, to another one-dram glass vial containing 4.0 mg CTAT. The vial was wrapped in aluminum foil to shield the contents from light and stirred for 18 h at room temperature. The entire 1 mL was then purified according to the 1 mL crude vesicle purification procedure outlined below, yielding 1.5 mL of purified vesicles.

Synthesis of 0.5-10 kbp DNA-ladder vesicles with and without spermine: A one-dram glass vial was charged with 25 μL of a DNA ladder stock solution (New England Biolabs, N3232S, 500 μg/mL) along with sterile stir bar. For formulations containing spermine, 2 μL of either 7.6 μM or 76 μM spermine in water was added and stirred for 20 min at room temperature. Next, 100 μL PBS 1× along with 150 μL of a 10 g/L SDBS solution in PBS 1× were added (final volume 250 μL) and allowed to stir for 1 h at room temperature. To a second one-dram vial was added 100 μL of a 10 g/L CTAT solution in MeOH. The MeOH was then removed at 60° C. on a rotovap, leaving a white film. The 250 μL SDBS/DNA/spermine solution, along with the stir bar, was transferred to the vial containing CTAT and stirred at room temperature for 18 h.

On the following day 5 μL CaCl₂) (25 mM), 2.5 μL MgCl₂ (250 mM), and 2.5 μL Turbo DNase (Thermo Fisher, AM2238, 2 U/μL) was added to the crude vesicle mixture and incubated at 37° C. for 2 h. After DNase treatment, 200 μL of the crude vesicle mixture was removed and purified according to the 200 μL vesicle purification procedure outlined below, yielding 300 μL of purified vesicles.

Synthesis of pUC18 plasmid vesicles: A one-dram glass vial was charged with 160 μL of a 10 mg/mL CTAT solution in MeOH followed by 20 μL of a 500 μg/mL pUC18 plasmid (2686 bp) solution in water. The solvent was removed on a rotovap at 50° C. leaving behind a white CTAT/DNA film. Next, 240 μL of a 10 mg/mL SDBS solution in PBS 1× was added along with a small stir bar and the mixture was stirred for 18 h at room temperature.

On the following day, 200 μL of the crude vesicles were transferred to a 2.0 mL Eppendorf tube. To the tube was also added 4 μL CaCl₂), 2 μL 250 mM MgCl₂, and 2 Turbo DNase (Thermo Fisher, AM2238, 2 U/μL). The reaction was incubated at 37° C. for 2 h then the entire 200 μL volume was purified according to the purification of 200 μL crude vesicles procedure outlined below, yielding 300 μL of purified vesicles.

Synthesis of pGFP vesicles: A one-dram glass vial was charged with 160 of a 10 mg/mL CTAT solution in MeOH followed by 20 μL of a 500 μg/mL GFP plasmid (Amaxa pmaxGFP, 3486 bp) solution in 10 mM Tris pH 8.0. The solvent was removed on a rotovap at 50° C. leaving behind a white CTAT/DNA film. Next, 240 μL of a 10 mg/mL SDBS solution in PBS 1× was added along with a small stir bar and the mixture was stirred for 18 h at room temperature.

On the following day, 200 μL of the crude vesicles were transferred to a 2.0 mL Eppendorf tube. To the tube was also added 4 μL 25 mM CaCl₂), 2 μL 250 mM MgCl₂, and 2 μL Turbo DNase (Thermo Fisher, AM2238, 2 U/μL). The reaction was incubated at 37° C. for 1 h then the entire 200 μL volume was purified according to the purification of 200 crude vesicles procedure outlined below, yielding 300 μL of purified vesicles.

Synthesis of linearized DNA vesicles: A 2 mL Eppendorf tube was charged with 25 μL of a 670 μg/mL stock solution of linearized SPIKE pDNA (see linearization procedure below). Next was added 600 μL 10 mg/mL SDBS in PBS 1× along with an additional 375 μL PBS 1×. The solution was allowed to incubate at room temperature for 1 h. The SDBS/DNA solution (1.0 mL) was then transferred to a one-dram glass vial containing 4.0 mg CTAT. The mixture was stirred at room temperature for 18 h.

On the following day, 1.0 mL of the crude vesicles were combined with 20 μL 25 mM CaCl₂), 10 μL 250 mM MgCl₂, and 5 μL Turbo DNase (Thermo Fisher, AM2238, 2 U/μL). The reaction was incubated at 37° C. for 3 h then the 1.0 mL volume was purified according to the purification of 1.0 mL crude vesicles procedure outlined below, yielding 300 of purified vesicles.

Crude Vesicle Purification.

Plastic columns were decontaminated with RNaseZap and then rinsed with Milli-Q water.

Purification of 1 mL crude vesicles: A gravity column was packed with degassed Sepharose CL-2B (Sigma, CL2B300) to 5.0 cm height and 1.5 cm diameter. The gel was flushed with two column-volumes of PBS 1×. Next, 1.0 mL of crude vesicle suspension was loaded onto the column and gravity fed into the gel. 2.0 mL of PBS 1× eluent was then added to the column and the fractions discarded. 1.5 mL of PBS 1× was then added to the column and the fractions collected to yield purified opaque light blue vesicles. The column was cleaned with 2.0 mL of 0.1% Triton X-100 in water followed by two column volumes of PBS 1×.

Purification of 200 μL crude vesicles: A small 3 mL gravity column was packed with degassed Sepharose CL-2B (Sigma, CL2B300) to 17 mm height and 9 mm diameter. The gel was flushed with two column-volumes of PBS 1×. Next, 200 μL of crude vesicle suspension was loaded onto the column and gravity fed into the gel. 200 μL of PBS 1× eluent was then added to the column and the fractions discarded. 300 μL of PBS 1× was then added to the column and the fractions collected to yield purified opaque light blue vesicles. The column was cleaned with 400 μL of 0.1% Triton X-100 in water followed by two column volumes of PBS 1×.

Fluorescein Labeled DNA and Quenched DNA Synthesis and Purification: DNA oligonucleotides 5′-d(ACC-CTA-CGT-ATC-GGT-CAG-TC-Fluorescein (SEQ ID NO:3)) and 5′-d(BlackHoleQuencher2-GAC-TGA-CCG-ATA-CGT-AGG-GT (SEQ ID NO:4)) were synthesized DMT-off on a 1 μmol scale using standard phosphoramidite chemistry on an Expedite 8909 Nucleic Acid Synthesizer (PerSeptive Biosystems, Framingham, Mass.) with reagents from Glen Research (Sterling, Va.). Oligonucleotides were deprotected with 30% ammonium hydroxide, purified by denaturing 20% (19:1) acrylamide/bis-acrylamide, 7 M urea gel electrophoresis, and dialyzed against pure water.

DNase I Protection Assay: Intact DNA duplex loaded vesicles were added to reaction buffer containing 2.5 mM magnesium chloride, 0.5 mM calcium chloride, and 1× PBS. DNase I (Thermo Fisher Scientific, Waltham, Mass.) was added to a final concentration of 0.005 U/μL and the reaction was monitored by fluorescence (excitation at 494 nm, emission 521 nm) collected on SpectraMax M5 Microplate Reader (Molecular Devices, San Jose, Calif.) over 3 h at 25° C. or 37° C. Broken vesicles were prepared by 3 freeze thaw cycles of 10 min at −80° C. and 2 min at RT. Following the freeze thaw procedure, the samples equilibrated at RT for 30 min and were used in the assay as described above.

Linearization of SPIKE pDNA. To a 1.5 mL Eppendorf tube was added 23 μL of a 1.8 μg/μL stock solution of SPIKE plasmid (BEI Cat #52490, 9230 bp). Next was added 8 μL 20 U/μL XbaI (NEB Cat #R0145S), 5 μL CutSmart buffer (NEB Cat #B7204), and 14 μL Milli-Q water. The reaction was incubated at 37° C. for 4 h. Next was added 250 μL Milli-Q water along with 500 μL 24:24:1 v/v phenol:chloroform:isoamyl alcohol (ThermoFisher Cat #15593031). The mixture was vortexed for 10 s, briefly centrifuged to separate layers, and the aqueous layer was collected. The organic phase was extracted with an additional 300 μL water and combined with the other aqueous fraction (total volume ˜600 μL). The extracted DNA solution was split into 2×300 μL aliquots, and each combined with 600 μL EtOH then placed in a −20° C. freezer for 20 h.

On the following day, the precipitated DNA was centrifuged at 15,000 rpm for 20 minutes at 4° C. then decanted. The pellet was gently washed with an additional 500 EtOH and dried on a vacufuge at 40° C. for 10 min. The pellets were dissolved in 15 Milli-Q water each, and combined to provide a 670 μg/mL stock (measured on a NanoDrop spectrophotometer) of linearized SPIKE pDNA, which was stored at −20° C.

DNase Challenge of 13 nt ssDNA Vesicles.

Without disruption: A 16 μL aliquot was removed from the DNA-vesicle solution and combined with 0.5 U 51 Nuclease and 4 μL 5× reaction buffer (ThermoFisher Cat #EN0321). The sample was allowed to react at room temperature for 0.5 h. It was then combined with 20 μL 2× Laemmli buffer containing DTT, heated at 100° C. for 5 min, and analyzed by PAGE according to the conditions in the section below.

With disruption: A 16 μL aliquot was removed from the DNA-vesicle solution and combined with 0.5 U 51 Nuclease and 4 μL 5× reaction buffer (ThermoFisher Cat #EN0321). The sample was frozen at −80° C. for 10 minutes followed by thawing gently in warm water. This process was repeated two more times, and then allowed to incubate at room temperature for 0.5 h. It was then combined with 20 μL 2× Laemmli buffer containing DTT, heated at 100° C. for 5 min, and analyzed by PAGE according to the conditions in the section below.

RNase Challenge of 21 bp siRNA Vesicles.

Without disruption: A 2 μL aliquot of the siRNA vesicles were combined with 7 μL PBS 1× and 1 μL 100 mg/mL RNase (Sigma, R5500). The sample was then combined with 10 μL 2× Laemmli buffer containing DTT, heated at 100° C. for 5 min, and analyzed by PAGE according to the conditions in the section below.

With disruption: A 2 μL aliquot of the siRNA vesicles were combined with 7 μL PBS 1× and 1 μL 100 mg/mL RNase (Sigma, R5500). The sample was then frozen at −80° C. for 10 minutes followed by thawing gently in warm water. This process was repeated two more times. The sample was then combined with 10 μL 2× Laemmli buffer containing DTT, heated at 100° C. for 5 min, and analyzed by PAGE according to the conditions in the section below.

DNase Challenge of Plasmid and Ladder Vesicles.

Without disruption: A 50 μL aliquot was combined with 1 μL 25 mM CaCl₂), 0.5 μL 250 mM MgCl₂, and 1 μL Turbo DNase (Thermo Fisher, AM2238, 2 U/μL). The sample was incubated at 37° C. for 1 h. The sample was then heated at 100° C. for 10 min to deactivate the DNase and dried on a vacufuge and reconstituted with 20 μL 0.5 g/mL HPβCD and 10 μL Milli-Q water. The sample was vortexed and briefly heated to ensure solubilization. Next was added 6 μL 6× loading dye and analyzed on an agarose gel according to the procedure below.

With disruption: A 50 μL aliquot was combined with 1 μL 25 mM CaCl₂), 0.5 μL 250 mM MgCl₂, 1 μL Turbo DNase (Thermo Fisher, AM2238, 2 U/μL) and 20 μL 0.5 g/mL HPβCD. The sample was incubated at 37° C. for 1 h. The sample was then heated at 100° C. for 10 min to deactivate the DNase and dried on a vacufuge and reconstituted with 30 Milli-Q water. The sample was vortexed and briefly heated to ensure solubilization. Next was added 6 μL 6× loading dye and analyzed on an agarose gel according to the procedure below.

Polyacrylamide Gel Conditions.

Vesicles containing 13 nt ssDNA and 21 bp siRNA were analyzed by PAGE.

Sample Prep: Vesicles were diluted 5× in PBS 1× (2 μL DNA-vesicles+8 μL PBS 1×). 10 μL 2× Laemmli sample buffer containing DTT was then added, and the sample heated at 100° C. for 5 min. The sample was then vortexed briefly and centrifuged to collect the solution in the bottom of the tube.

PAGE Gel: The samples (15 μL) were loaded onto a 20% polyacrylamide gel. The running buffer was 7 M urea in TBE 1×. The gel was run at constant voltage (180 V) until the dye reached the bottom of the gel (˜2 h).

Agarose Gel Conditions.

Vesicles containing 0.5-10 kbp ladder, pUC18, pGFP, and linearized SPIKE plasmid were analyzed on agarose gel.

Sample Prep: An Eppendorf tube was charged with 50 μL vesicle sample and 20 μL 0.5 g/mL HPβCD. The sample was dried on a vacufuge at 60° C. (˜10 min) and reconstituted with 30 μL Milli-Q water. The sample was vortexed and briefly heated to solubilize the residue. Then 6 μL 6× gel loading dye was added and the sample vortexed briefly and centrifuged to collect the solution in the bottom of the tube.

Agarose Gel: The agarose gel was comprised of 1 wt. % agarose in 1×TBE buffer with 3 μL ethidium bromide stain. The gel was then run at constant voltage (100 V) with 1×TBE as the running buffer (˜1 h), or until the dye reached the bottom of the gel. In the case of linearized DNA vesicles, the gel was re-stained with SYBR Gold (ThermoFisher, Cat #S11494).

### Example 2

This example provides a description of methods of the present disclosure.

To two separate, oven-dried, 1-dram vial, 2 μL of a 76 μM spermine solution was added with 25 μL of a 1 KB DNA Ladder from New England Biolabs [Catalog #N3232S] and a sterile, magnetic stir-bar. The solution was mixed at medium speed for 15 minutes at room temperature. These steps were repeated but using a 7.6 μM spermine solution instead. To one of the vials, 223 μL of pre-formed, crude vesicles was added and left to stir for 18 hours. The other vial had 150 μL of a 10 mg/mL SDBS in PBS stock added and let stir for 1 hour. At the same time, 100 μL of a 10 mg/mL CTAT stock in methanol was added to a separate, oven-dried, 1-dram vial. This solution was then placed on a rotary evaporator until completely dry. The SDBS/spermine/DNA solution was then added to the dry CTAT vial and left to stir for 18 hours at room temperature. As a control, 25 μL of the DNA Ladder was added to SDBS then added to dry CTAT and left to stir for 18 hours, as previously outlined. At this point, to recap, there should be five solutions stirring: 1) 76 μM spermine/DNA+Crude Vesicles 2) 7.6 μM spermine/DNA+Crude Vesicles 3) 76 μM spermine/DNA Vesicles 4) 7.6 μM spermine/DNA Vesicles 5) DNA Vesicles.

The following day, 5 μL of 25 mM CaCl2, 2.5 μL of 250 mM MgCl₂, and 2.5 μL of Turbo DNAse from Thermo Fisher [Catalog #AM2238] was added to each of the five solutions and left to incubate at 37° C. for 120 minutes. After the samples were removed from the incubator, they were purified on an approximately 7 mm tall×1 mm wide separation column from Marvelgent Biosciences [Catalog #11-0258] packed with 1.5 mm of Sepharose CL2B gel from Sigma [Catalog #CL2B300], sandwiched between two 20 μm filters. To begin the purification process, approximately 1 mL of an RNA-Zap spray was added to the column to digest any potential nucleases that may be present. The RNA-Zap solution was then flushed out with two columns worth of Nuclease-Free PBS. Then, 200 μL of a vesicle solution was loaded onto the column. Once the solution ceases to elute, 200 μL of Nuclease-free PBS was added. Finally, once that finishes eluting, an oven-dried 1-dram vial is placed underneath the column and 300 μL of Nuclease-free PBS was added to the column. The resulting eluant collected is the purified vesicle fraction.

To measure the average size of the vesicles, 100 μL of the solution was taken and diluted to 1 mL with water and analyzed using dynamic light scattering (DLS).

From each of the purified vesicle samples, 50 μL aliquots were taken. One aliquot is set aside, the second aliquot has 1 μL of 25 mM CaCl₂), 0.5 μL of 250 mM MgCl₂, and 0.5 μL of Turbo DNAse added. A third aliquot has the same DNAse treatment as well as an additional 20 μL of a 0.5 g/mL solution of (2-hydroxypropyl)-β-cyclodextrin (HPβCD) to disrupt the vesicle formation and expose the DNA ladder to the DNAse treatment. All the samples were placed in a 37° C. incubator for 120 minutes. Once removed, they were placed in a 100° C. heating block for 10 minutes to deactivate the DNAse. Next, all solutions were placed in a 60° C. vacufuge for 20 minutes until dry. All samples were reconstituted in 10 μL of water and 10 μL of 0.5 g/mL HPβCD to ensure micelles do not reform (if the sample already contained HPβCD, an additional 10 μL of water is substituted). Then, 4 μL of a 6× purple loading dye was added to each sample and vortexed to fully mix.

To prepare the gel, 1 g of agarose was boiled with 100 mL of 1×TBE buffer. Once it began to cool, 3 μL of ethidium bromide was added and swirled into solution. Before it cools completely, the solution was poured into a gel mold with a 10-lane well comb. After the gel solidified, it was placed in a gel box filled with 1×TBE buffer and the comb removed. The entirety of each sample, including a DNA ladder control lane (1 μL DNA Ladder+19 μL water+4 μL Dye), were loaded into the gel. The cover and wires were placed onto the gel box and set to 100 V for 3 hours. Once the gel finished running, it was removed and imaged using the imager software.

### Example 3

Empty vesicles vs. DOX-vesicles. 3 cell lines: MDA-MB-231: human breast cancer; MCF-7: human breast cancer; A549: non-small cell lung cancer.

| | 231 | | MCF-7 |
|---|---|---|---|
| | (breast) | A549 | (breast) |
| Vesicles | (ug/ml) | (ug/ml) | ug/ml |
| Dox Ves (n = 2) | 4.2 ± 0.6 | 3.6 ± 0.9 | 9.5 ± 0.7 |
| Empty Ves (n = 2) | 2.0 ± 0.3 | 4.2 ± 0.4 | 7.0 ± 1.2 |
| Dox (Free) n = 1 | 0.06 | 0.02 | NE# |

Although the present disclosure has been described with respect to one or more particular embodiments and/or examples, it will be understood that other embodiments and/or examples of the present disclosure may be made without departing from the scope of the present disclosure.

1. A catanionic surfactant vesicle comprising
a cationic surfactant and an anionic surfactant,
wherein the cationic surfactant is cetyltrimethylammonium tosylate (CTAT) and the anionic surfactant is sodium dodecylbenzene sulfonate (SDBS) and the SDBS and CTAT are present in a ratio of 60:40 to 80:20 (SDBS:CTAT, w/w);
the cationic surfactants and the anionic surfactants define a leaflet having inner leaflet and an outer leaflet and the inner leaflet defines a lumen; and
the catanionic surfactant vesicle at least partially encapsulates one or more small molecules and/or one or more siRNAs.

2. The catanionic surfactant vesicle according to claim 1, wherein the ratio of SDBS to CTAT is 65:35 to 70:30.

3. The catanionic surfactant vesicle according to claim 2, wherein the ratio of SDBS to CTAT is 65:35 or 70:30.

4. The catanionic surfactant vesicle according to claim 1, wherein the outer leaflet of the catanionic surfactant vesicle is functionalized with one or more conjugation groups, one or more cell surface receptor binders, one or more small molecules, one or more peptides and/or proteins, one or more carbohydrates, one or more glycans, one or more polysaccharides, one or more nucleic acid derivatives, one or more lipids, and/or one or more monoclonal antibodies.

5. The catanionic surfactant vesicle according to claim 4, wherein the one or more cell surface receptor binders are folate receptors.

6. The catanionic surfactant vesicle according to claim 4, wherein the one or more monoclonal antibodies are Herceptin, Rituximab, nivolumab, or a combination thereof.

7. The catanionic surfactant vesicle according to claim 4, wherein the one or more conjugation groups have an alkyne, an azide, a thiol, a disulfide, a maleimide, a thioester, or a cyanuril chloride derivative.

8. The catanionic surfactant vesicle according to claim 1, wherein the one or more small molecules is a chemotherapy drug, saccharide, antibiotic, or biologically active group.

9. The catanionic surfactant vesicle according to claim 8, wherein the chemotherapy drug is doxorubicin, cisplatin, carboplatin, pemetrexed, auristatin, maytansine, paclitaxel, camptothecin, vincristine, vinblastine, irinotecan, amphotericin B, salts thereof, or combinations thereof.

10. The catanionic surfactant vesicle according to claim 1, wherein the catanionic surfactant vesicle encapsulates at least 100 small molecules or siRNAs.

11. The catanionic surfactant vesicle according to claim 1, wherein the one or more small molecules and/or one or more siRNAs are partially encapsulated in the lumen and/or the leaflet.

12. A catanionic surfactant vesicle comprising
a cationic surfactant and an anionic surfactant,
wherein the cationic surfactant is cetyltrimethylammonium tosylate (CTAT) and the anionic surfactant is sodium dodecylbenzene sulfonate (SDBS) and the SDBS and CTAT are present in a ratio of 60:40 to 80:20 (SDBS:CTAT, w/w);
the cationic surfactants and the anionic surfactants define a leaflet having inner leaflet and an outer leaflet and the inner leaflet defines a lumen; and
the catanionic surfactant vesicle at least partially encapsulates one or more protein, one or more DNAs, and/or one or more RNAs.

13. The catanionic surfactant vesicle according to claim 12, further comprising polyamines and/or condensed polyamines.

14. The catanionic surfactant vesicle according to claim 13, wherein the polyamines are spermine.

15. The catanionic surfactant vesicle according to claim 12, wherein the one or more DNAs and/or one or more RNAs have 20 or more nucleobases or nucleobase pairs.

16. The catanionic surfactant vesicle according to claim 12, wherein the one or more RNAs are amino acid-encoding RNAs.

17. The catanionic surfactant vesicle according to claim 14, wherein the one or more DNAs are plasmids.

18. A composition comprising the catanionic surfactant vesicle according to claim 1 and a pharmaceutical carrier.

19. A vaccine composition comprising the catanionic surfactant vesicle according to claim 12 and a pharmaceutical carrier and, optionally, one or more adjuvants.

20. A method for prevention, treatment, amelioration, or management of an individual having cancer comprising administering one or more catanionic surfactant vesicles according to claim 1 or a composition thereof to the individual.

21. The method according to claim 20, wherein the cancer is hepatic cancer, colon cancer, rectal cancer, breast cancer, prostate cancer, skin cancer, head and neck cancer, lung cancer, gastric cancer, mesothelioma, melanoma, lymphoma, Barrett's esophagus, synovial sarcoma, cervical cancer, endometrial ovarian cancer, Wilm's tumor, bladder cancer, leukemia, or a combination thereof.

22. The method according to claim 20, wherein the outer leaflet of the one or more catanionic surfactant vesicles is functionalized with one or more conjugation groups, one or more cell surface receptor binders, one or more small molecules, one or more peptides and/or proteins, one or more carbohydrates, one or more glycans, one or more polysaccharides, one or more nucleic acid derivatives, one or more lipids, and/or one or more monoclonal antibodies.

23. A method for inducing an immune response in an individual comprising administering the vaccine composition according to claim 19, wherein following administration, the individual has an immune response to a virus.


SEQUENCE LISTING (4 sequences)

SEQ ID NO: 1 - DNA, 13 bp, synthetic construct
  source 1..13: mol_type = other DNA
ggacagctgggag

SEQ ID NO: 2 - RNA, 21 nt, synthetic construct
  source 1..21: mol_type = other RNA
gtgtatccaacacggatcctc

SEQ ID NO: 3 - DNA, 20 bp, synthetic construct
  source 1..20: mol_type = other DNA
  modified_base 20: mod_base = OTHER; note = Fluorescein is covalently attached to cytosine
accctacgtatcggtcagtc

SEQ ID NO: 4 - DNA, 20 bp, synthetic construct
  source 1..20: mol_type = other DNA
  modified_base 1: mod_base = OTHER; note = Black Hole Quencher 2 is covalently attached to guanine
gactgaccgatacgtagggt